(19) United States
(12) Patent Application Publication
(10) Pub. No.: US 2025/0050045 A1
(43) Pub. Date: Feb. 13, 2025

(54) ACCELEROMETER-BASED USER INTERFACE LEAKAGE DETECTION

(71) Applicant: ResMed Digital Health Inc., San Diego, CA (US)

(72) Inventor: Michael BRODERICK, Dublin (IE)

(21) Appl. No.: 18/797,938

(22) Filed: Aug. 8, 2024

Related U.S. Application Data
(60) Provisional application No. 63/519,101, filed on Aug. 11, 2023.

(51) Int. Cl. A61M 16/00; A61M 16/06; G01P 15/18

(52) U.S. Cl. CPC A61M 16/024; A61M 16/0683; G01P 15/18; A61M 2205/15; A61M 2205/3306; A61M 2230/63

(57) ABSTRACT

A method for analyzing user interface leakage includes receiving, at a computing device, motion data associated with orientation of a user interface worn by a user during a sleep session. The method also includes analyzing the motion data to identify leak data. The leak data indicative of at least one unintentional leak from the user interface. The method also includes generating a notification based at least in part on the leak data, the notification indicative of the presence of the at least one unintentional leak. The notification can provide guidance to reduce, minimize or eliminate the unintentional leak.

### CROSS-REFERENCE TO RELATED APPLICATIONS

This present application claims the benefit of U.S. Provisional Patent Application No. 63/519,101, filed Aug. 11, 2023 and entitled “ACCELEROMETER-BASED USER INTERFACE LEAKAGE DETECTION,” the disclosure of which is hereby incorporated by reference in its entirety.

### TECHNICAL FIELD

The present disclosure relates to respiratory therapy generally and more specifically to detecting and reducing user interface leakage in respiratory therapy.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. The efficacy of such therapies relies heavily on the use of a well-fit user interface. Different types of user interfaces are available to accommodate various users' physical features and personal preferences. Even after being properly adjusted initially, the fit of a user interface can be affected over time due to various factors, such as changes in the users' physical features (e.g., swelling or hair growth), intentional or unintentional adjustments to the user interface or related equipment, impact or damage to the user interface or related equipment, and general wear and tear of user interface components (e.g., cushions) over time.

As a result, user interfaces can leak pressurized air, thus reducing their efficacy. Further, air leaks can often lead to user discomfort and therapy non-compliance, such as if the user decides not to engage in the therapy due to the uncomfortable sensation or sound associated with the air leak. Air leaks are not generally visible to the user and some air leaks cannot be heard by a user. Users often have difficulty in locating and eliminating air leaks. Some users attempt to reduce or eliminate air leaks by tightening the straps of their user interface, which can result in overtightening that can cause discomfort and undesirable lines or marks on the user's face.

Some respiratory therapy devices are capable of detecting the existence of a leak downstream of the respiratory therapy device itself. However, detection of the existence of such a leak does not help inform where that leak may be located and does not help a user correct or mitigate such a leak. In fact, due to the difficulties associated with locating air leaks around a user interface, users may incorrectly conclude the air leak is occurring within the conduit and/or the respiratory therapy device itself, which may lead to unnecessary expense to the user and/or manufacturer.

Therefore, there is a need for improved techniques for detecting and locating air leaks associated with user interfaces. There is a need for an easy-to-use tool to guide the user in adjusting the user interface or accompanying equipment to reduce or minimize air leaks. There is a need for such a tool to help guide the user in achieving proper fit of a user interface. The present disclosure is directed to solving these and other problems.

### BRIEF SUMMARY

According to some implementations of the present disclosure, a method for analyzing user interface leakage includes receiving, at a computing device, motion data associated with orientation of a user interface worn by a user during a sleep session. The method further includes analyzing the motion data to identify leak data. The leak data is indicative of at least one unintentional leak from the user interface. The method further includes generating a notification based at least in part on the leak data, the notification indicative of the presence of the at least one unintentional leak.

According to some implementations of the present disclosure, a system includes one or more motion sensors coupled to a user interface worn by a user during a sleep session. The user interface is fluidly coupled to a respiratory therapy device for providing a flow of air from the respiratory therapy device to a respiratory system of the user. The system further includes one or more processors and a non-transitory computer-readable storage medium containing instructions which, when executed on the one or more processors, cause the one or more processors to perform operations including receiving, at a computing device, motion data associated with orientation of the user interface. The operations further include analyzing the motion data to identify leak data. The leak data is indicative of at least one unintentional leak from the user interface. The operations further include generating a notification based at least in part on the leak data, the notification indicative of the presence of the at least one unintentional leak.

The above summary is not intended to represent each implementation or every aspect of the present disclosure. Additional features and benefits of the present disclosure are apparent from the detailed description and figures set forth below.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3A is a perspective view of a respiratory therapy device of the system of FIG. 1, according to some implementations of the present disclosure.

FIG. 3B is a perspective view of the respiratory therapy device of FIG. 3A illustrating an interior of a housing, according to some implementations of the present disclosure.

FIG. 4A is a perspective view of a user interface, according to some implementations of the present disclosure.

FIG. 4B is an exploded view of the user interface of FIG. 4A, according to some implementations of the present disclosure.

FIG. 5A is a perspective view of a user interface, according to some implementations of the present disclosure.

FIG. 5B is an exploded view of the user interface of FIG. 5A, according to some implementations of the present disclosure.

FIG. 6A is a perspective view of a user interface, according to some implementations of the present disclosure.

FIG. 6B is an exploded view of the user interface of FIG. 6A, according to some implementations of the present disclosure.

FIG. 7 illustrates an exemplary timeline for a sleep session, according to some implementations of the present disclosure.

FIG. 8 illustrates an exemplary hypnogram associated with the sleep session of FIG. 7, according to some implementations of the present disclosure.

FIG. 9 is a flow chart depicting a process for analyzing user interface leak, according to certain aspects of the present disclosure.

FIG. 10 is a flow chart depicting a process for analyzing user interface leak using features extracted from motion data, according to certain aspects of the present disclosure.

FIG. 11 is a set of charts depicting collected data from a user interface exhibiting unintentional leaks, according to certain aspects of the present disclosure.

FIG. 12 is a set of charts depicting collected data from a user interface exhibiting unintentional leaks, according to certain aspects of the present disclosure.

FIG. 13 is a set of charts depicting frequency-domain acceleration data associated with unintentional leaks at different locations with respect to a user interface, according to certain aspects of the present disclosure.

FIG. 14 is a chart depicting classification of unintentional leaks based on two features, using a user-interface-mounted sensor, according to certain aspects of the present disclosure.

FIG. 15 is a set of charts depicting frequency-domain acceleration data and frequency-domain rotational velocity data associated with an unintentional leak as detected from an accelerometer and gyroscope located on a frame of a user interface, according to certain aspects of the present disclosure.

FIG. 16 is a set of charts depicting frequency-domain acceleration data and frequency-domain rotational velocity data associated with an unintentional leak as detected from an accelerometer and gyroscope located on an elbow connector of a user interface, according to certain aspects of the present disclosure.

FIG. 17 is a set of charts depicting frequency-domain acceleration data and frequency-domain rotational velocity data associated with an unintentional leak as detected from an accelerometer and gyroscope located on a conduit cuff coupled to a user interface, according to certain aspects of the present disclosure.

FIG. 18 is a chart depicting frequency-domain rotational velocity data used to differentiate straps of a user interface likely to be causing an unintentional leak, according to certain aspects of the present disclosure.

FIG. 19 is a set of charts depicting various features of motion data used to differentiate straps of a user interface likely to be causing an unintentional leak, according to certain aspects of the present disclosure.

FIG. 20 is a chart depicting classification of unintentional leaks based on two features, using a conduit-cuff-mounted sensor, according to certain aspects of the present disclosure.

FIG. 21 is a chart depicting classification of unintentional leaks based on two features, according to certain aspects of the present disclosure.

FIG. 22 is a set of confusion matrix charts depicting classification accuracy when different numbers of features are used to classify the unintentional leak, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of Sleep Disordered Breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Certain aspects of the present disclosure can be implemented as a system, a method, and/or a computer program product. The computer program product may include a computer readable storage medium (or media) (e.g., a memory device) having computer readable program instructions thereon for causing a processor (e.g., a control system) to carry out aspects of the present disclosure.

Certain aspects of the present disclosure enable the identification and characterization of unintentional leaks of a user interface from motion data associated with the user interface. Certain aspects of the present disclosure can advantageously identify and characterize these unintentional leaks even while the user is asleep, thus permitting unintentional leaks to be better understood during sleep. Certain aspects of the present disclosure enable the user to be notified of unintentional leaks and/or be provided with instructions for corrective action to take. For example, the user may be notified to tighten one of multiple straps of the user interface to correct an otherwise unnoticed unintentional leak that may be affecting the efficacy of the user's respiratory therapy.

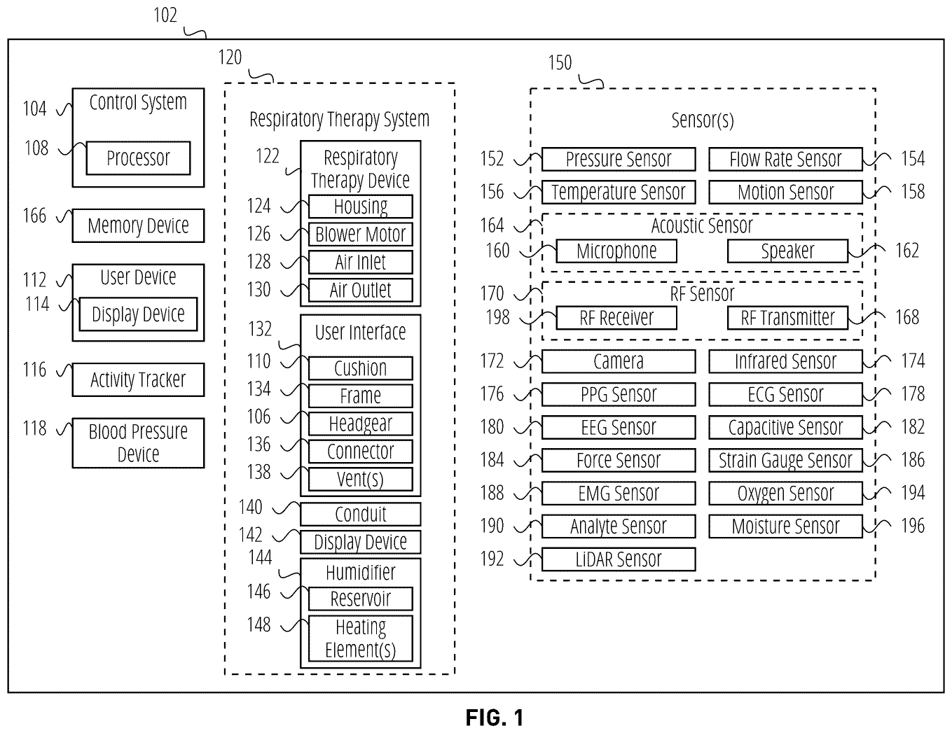

FIG. 1 is a functional block diagram of a system 102, according to some implementations of the present disclosure. The system 102 includes a respiratory therapy system 120, a control system 104, one or more sensors 150, a user device 112, and an activity tracker 116.

The respiratory therapy system 120 includes a respiratory pressure therapy (RPT) device 122 (referred to herein as respiratory therapy device 122), a user interface 132 (also referred to as a mask or a patient interface), a conduit 140 (also referred to as a tube or an air circuit), a display device 142, and a humidifier 144. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

The respiratory therapy system 120 can be used to treat a user. The user interface 132 can be worn by the user during a sleep session. The respiratory therapy system 120 generally aids in increasing the air pressure in the throat of the user to aid in preventing the airway from closing and/or narrowing during sleep. The respiratory therapy device 122 can be positioned nearby, such as on a nightstand adjacent to where the user is engaging in the sleep session, or more generally, on any surface or structure that is generally adjacent to the user and/or the user's sleeping surface (e.g., bed).

The respiratory therapy device 122 is generally used to generate pressurized air that is delivered to a user (e.g., using one or more motors that drive one or more compressors). The respiratory therapy device 122 includes a housing 124, a blower motor 126, an air inlet 128, and an air outlet 130. In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 generates a variety of different air pressures within a predetermined range. For example, the respiratory therapy device 122 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, and the like. The respiratory therapy device 122 can also deliver pressurized air at a predetermined flow rate between, for example, about −20 L/min and about 150 L/min, while maintaining a positive pressure (relative to the ambient pressure).

The user interface 132 engages a portion of the user's face and delivers pressurized air from the respiratory therapy device 122 to the user's airway to aid in preventing the airway from narrowing and/or collapsing during sleep. This delivery of pressurized air may also increase the user's oxygen intake during sleep. Generally, the user interface 132 engages the user's face such that the pressurized air is delivered to the user's airway via the user's mouth, the user's nose, or both the user's mouth and nose. Together, the respiratory therapy device 122, the user interface 132, and the conduit 140 form an air pathway fluidly coupled with an airway of the user. Depending upon the therapy to be applied, the user interface 132 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface 132 not include a seal sufficient to facilitate delivery to the airways of a supply of gas at a positive pressure of about 10 cmH₂O.

The user interface 132 can include, for example, a cushion 110, a frame 134, a headgear 106, connector 136, and one or more vents 138. The cushion 110 and the frame 134 define a volume of space around the mouth and/or nose of the user. When the respiratory therapy system 120 is in use, this volume space receives pressurized air (e.g., from the respiratory therapy device 122 via the conduit 140) for passage into the airway(s) of the user. The headgear 106 is generally used to aid in positioning and/or stabilizing the user interface 132 on a portion of the user (e.g., the face), and along with the cushion 110 (which, for example, can comprise silicone, plastic, foam, etc.) aids in providing a substantially air-tight seal between the user interface 132 and the user. In some implementations the headgear 106 includes one or more straps (e.g., including hook and loop fasteners). The connector 136 is generally used to couple (e.g., connect and fluidly couple) the conduit 140 to the cushion 110 and/or frame 134. Alternatively, the conduit 140 can be directly coupled to the cushion 110 and/or frame 134 without the connector 136. The vent 138 can be used for permitting the escape of carbon dioxide and other gases exhaled by the user. The user interface 132 generally can include any suitable number of vents 138 (e.g., one, two, five, ten, etc.).

The conduit 140 (also referred to as an air circuit or tube) allows the flow of air between components of the respiratory therapy system 120, such as between the respiratory therapy device 122 and the user interface 132. In some implementations, there can be separate limbs of the conduit 140 for inhalation and exhalation. In other implementations, a single limb conduit is used for both inhalation and exhalation.

The conduit 140 includes a first end that is coupled to the air outlet 130 of the respiratory therapy device 122. The first end can be coupled to the air outlet 130 of the respiratory therapy device 122 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 122 to power the one or more heating elements of the conduit 140. For example, the electrical contact can be electrically coupled to an electrical contact of the air outlet 130 of the respiratory therapy device 122. In this example, electrical contact of the conduit 140 can be a male connector and the electrical contact of the air outlet 130 can be female connector, or, alternatively, the opposite configuration can be used.

The display device 142 is generally used to display image(s) including still images, video images, or both and/or information regarding the respiratory therapy device 122. For example, the display device 142 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user, etc.). In some implementations, the display device 142 acts as a human-machine interface (HMI) that includes a graphic user interface (GUI) configured to display the image(s) as an input interface. The display device 142 can be a light emitting diode (LED) display, an organic LED (OLED) display, a liquid crystal display (LCD) display, or the like. The input interface can be, for example, a touchscreen or touch-sensitive substrate, a mouse, a keyboard, or any sensor system configured to sense inputs made by a human user interacting with the respiratory therapy device 122.

The humidifier 144 is coupled to or integrated in the respiratory therapy device 122 and includes a reservoir 146 for storing water that can be used to humidify the pressurized air delivered from the respiratory therapy device 122. The humidifier 144 includes a one or more heating elements 148 to heat the water in the reservoir to generate water vapor. The humidifier 144 can be fluidly coupled to a water vapor inlet of the air pathway between the blower motor 126 and the air outlet 130, or can be formed in-line with the air pathway between the blower motor 126 and the air outlet 130. For example, air may flow from the air inlet 128 through the blower motor 126, and then through the humidifier 144 before exiting the respiratory therapy device 122 via the air outlet 130.

While the respiratory therapy system 120 has been described herein as including each of the respiratory therapy device 122, the user interface 132, the conduit 140, the display device 114, and the humidifier 144, more or fewer components can be included in a respiratory therapy system 120 according to implementations of the present disclosure. For example, a first alternative respiratory therapy system includes the respiratory therapy device 122, the user interface 132, and the conduit 140. As another example, a second alternative system includes the respiratory therapy device 122, the user interface 132, and the conduit 140, and the display device 142. Thus, various respiratory therapy systems can be formed using any portion or portions of the components shown and described herein and/or in combination with one or more other components.

The control system 104 includes one or more processors 108 (hereinafter, processor 108). The control system 104 is generally used to control (e.g., actuate) the various components of the system 102 and/or analyze data obtained and/or generated by the components of the system 102. The processor 108 can be a general or special purpose processor or microprocessor. While one processor 108 is illustrated in FIG. 1, the control system 104 can include any number of processors (e.g., one processor, two processors, five processors, ten processors, etc.) that can be in a single housing, or located remotely from each other. The control system 104 (or any other control system) or a portion of the control system 104 such as the processor 108 (or any other processor(s) or portion(s) of any other control system), can be used to carry out one or more steps of any of the methods described and/or claimed herein. The control system 104 can be coupled to and/or positioned within, for example, a housing of the user device 112, a portion (e.g., the respiratory therapy device 122) of the respiratory therapy system 120, and/or within a housing of one or more of the sensors 150. The control system 104 can be centralized (within one such housing) or decentralized (within two or more of such housings, which are physically distinct). In such implementations including two or more housings containing the control system 104, the housings can be located proximately and/or remotely from each other.

The memory device 166 stores machine-readable instructions that are executable by the processor 108 of the control system 104. The memory device 166 can be any suitable computer readable storage device or media, such as, for example, a random or serial access memory device, a hard drive, a solid state drive, a flash memory device, etc. While one memory device 166 is shown in FIG. 1, the system 102 can include any suitable number of memory devices 166 (e.g., one memory device, two memory devices, five memory devices, ten memory devices, etc.). The memory device 166 can be coupled to and/or positioned within a housing 124 of a respiratory therapy device 122 of the respiratory therapy system 120, within a housing of the user device 112, within a housing of one or more of the sensors 150, or any combination thereof. Like the control system 104, the memory device 166 can be centralized (within one such housing) or decentralized (within two or more of such housings, which are physically distinct).

In some implementations, the memory device 166 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 108 and/or memory device 166 can receive data (e.g., physiological data and/or audio data) from the one or more sensors 150 such that the data for storage in the memory device 166 and/or for analysis by the processor 108. The processor 108 and/or memory device 166 can communicate with the one or more sensors 150 using a wired connection or a wireless connection (e.g., using a radio-frequency (RF) communication protocol, a Wi-Fi communication protocol, a Bluetooth communication protocol, over a cellular network, etc.). In some implementations, the system 102 can include an antenna, a receiver (e.g., an RF receiver), a transmitter (e.g., an RF transmitter), a transceiver, or any combination thereof. Such components can be coupled to or integrated a housing of the control system 104 (e.g., in the same housing as the processor 108 and/or memory device 166), or the user device 112.

The one or more sensors 150 include a pressure sensor 152, a flow rate sensor 154, temperature sensor 156, a motion sensor 158, a microphone 160, a speaker 162, a radio-frequency (RF) RF receiver 198, an RF transmitter 168, a camera 172, an infrared sensor 174, a photoplethysmogram (PPG) PPG sensor 176, an electrocardiogram (ECG) ECG sensor 178, an electroencephalography (EEG) EEG sensor 180, a capacitive sensor 182, a force sensor 184, a strain gauge sensor 186, an electromyography (EMG) EMG sensor 188, an oxygen sensor 194, an analyte sensor 190, a moisture sensor 196, a LiDAR sensor 192, or any combination thereof. Generally, each of the one or more sensors 150 is configured to output sensor data that is received and stored in the memory device 166 or one or more other memory devices.

While the one or more sensors 150 are shown and described as including each of the pressure sensor 152, the flow rate sensor 154, the temperature sensor 156, the motion sensor 158, the microphone 160, the speaker 162, the RF receiver 198, the RF transmitter 168, the camera 172, the infrared sensor 174, the PPG sensor 176, the ECG sensor 178, the EEG sensor 180, the capacitive sensor 182, the force sensor 184, the strain gauge sensor 186, the EMG sensor 188, the oxygen sensor 194, the analyte sensor 190, the moisture sensor 196, and the LiDAR sensor 192, more generally, the one or more sensors 150 can include any combination and any number of each of the sensors described and/or shown herein. Any number of the one or more sensors 150 can be integrated into any suitable housing, such as a unique housing for that sensor, a shared housing with that sensor and another sensor, a housing 124 of the respiratory therapy device 122, a different housing of the respiratory therapy system 120, a housing of control system 104, a housing of the user device 112, a housing of an activity tracker 116, a housing of a blood pressure device 118, or the like. In some case a sensor 150 can be incorporated across multiple housings.

As described herein, the system 102 generally can be used to generate physiological data associated with a user (e.g., a user of the respiratory therapy system 120) during a sleep session. The physiological data can be analyzed to generate one or more sleep-related parameters, which can include any parameter, measurement, etc. related to the user during the sleep session. The one or more sleep-related parameters that can be determined for the user during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user, temperature, EEG activity, EMG activity, arousal, snoring, choking, coughing, whistling, wheezing, or any combination thereof.

The one or more sensors 150 can be used to generate, for example, physiological data, audio data, or both. Physiological data generated by one or more of the sensors 150 can be used by the control system 104 to determine a sleep-wake signal associated with the user during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 150, are described in, for example, WO 2014/047310, U.S. Patent Pub. No. 2014/0088373, WO 2017/132726, WO 2019/122413, WO 2019/122414, and U.S. Patent Pub. No. 2020/0383580, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 150 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 132), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 150 can also be used to determine a respiration signal associated with a user during a sleep session. The respiration signal is generally indicative of respiration or breathing of the user during the sleep session. The respiration signal can be indicative of and/or analyzed to determine (e.g., using the control system 104) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 122, or any combination thereof. The one or more events can include snoring, apneas, central apneas, obstructive apneas, mixed apneas, hypopneas, a mask leak (e.g., from the user interface 132), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 152 outputs pressure data that can be stored in the memory device 166 and/or analyzed by the processor 108 of the control system 104. In some implementations, the pressure sensor 152 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 152 can be coupled to or integrated in the respiratory therapy device 122. The pressure sensor 152 can be, for example, a capacitive sensor, an electromagnetic sensor, a piezoelectric sensor, a strain-gauge sensor, an optical sensor, a potentiometric sensor, or any combination thereof.

The flow rate sensor 154 outputs flow rate data that can be stored in the memory device 166 and/or analyzed by the processor 108 of the control system 104. Examples of flow rate sensors (such as, for example, the flow rate sensor 154) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 122, an air flow rate through the conduit 140, an air flow rate through the user interface 132, or any combination thereof. In such implementations, the flow rate sensor 154 can be coupled to or integrated in the respiratory therapy device 122, the user interface 132, or the conduit 140. The flow rate sensor 154 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 154 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 152 can be used to determine a blood pressure of a user.

The temperature sensor 156 outputs temperature data that can be stored in the memory device 166 and/or analyzed by the processor 108 of the control system 104. In some implementations, the temperature sensor 156 generates temperatures data indicative of a core body temperature of the user, a skin temperature of the user, a temperature of the air flowing from the respiratory therapy device 122 and/or through the conduit 140, a temperature in the user interface 132, an ambient temperature, or any combination thereof. The temperature sensor 156 can be, for example, a thermocouple sensor, a thermistor sensor, a silicon band gap temperature sensor or semiconductor-based sensor, a resistance temperature detector, or any combination thereof.

The motion sensor 158 outputs motion data that can be stored in the memory device 166 and/or analyzed by the processor 108 of the control system 104. The motion sensor 158 can be used to detect movement of the user during the sleep session, and/or detect movement of any of the components of the respiratory therapy system 120, such as the respiratory therapy device 122, the user interface 132, or the conduit 140. The motion sensor 158 can include one or more inertial sensors, such as accelerometers, gyroscopes, and magnetometers. In some implementations, the motion sensor 158 alternatively or additionally generates one or more signals representing bodily movement of the user, from which may be obtained a signal representing a sleep state of the user; for example, via a respiratory movement of the user. In some implementations, the motion data from the motion sensor 158 can be used in conjunction with additional data from another one of the sensors 150 to determine the sleep state of the user.

The microphone 160 outputs sound and/or audio data that can be stored in the memory device 166 and/or analyzed by the processor 108 of the control system 104. The audio data generated by the microphone 160 is reproducible as one or more sound(s) during a sleep session (e.g., sounds from the user). The audio data form the microphone 160 can also be used to identify (e.g., using the control system 104) an event experienced by the user during the sleep session, as described in further detail herein. The microphone 160 can be coupled to or integrated in the respiratory therapy device 122, the user interface 132, the conduit 140, or the user device 112. In some implementations, the system 102 includes a plurality of microphones (e.g., two or more microphones and/or an array of microphones with beamforming) such that sound data generated by each of the plurality of microphones can be used to discriminate the sound data generated by another of the plurality of microphones.

The speaker 162 outputs sound waves that are audible to a user of the system 102. The speaker 162 can be used, for example, as an alarm clock or to play an alert or message to the user (e.g., in response to an event). In some implementations, the speaker 162 can be used to communicate the audio data generated by the microphone 160 to the user. The speaker 162 can be coupled to or integrated in the respiratory therapy device 122, the user interface 132, the conduit 140, or the user device 112.

The microphone 160 and the speaker 162 can be used as separate devices. In some implementations, the microphone 160 and the speaker 162 can be combined into an acoustic sensor 164 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 162 generates or emits sound waves at a predetermined interval and the microphone 160 detects the reflections of the emitted sound waves from the speaker 162. The sound waves generated or emitted by the speaker 162 have a frequency that is not audible to the human ear (e.g., below 20 Hz or above around 18 kHz) so as not to disturb the sleep of the user or the bed partner. Based at least in part on the data from the microphone 160 and/or the speaker 162, the control system 104 can determine a location of the user and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 150 include (i) a first microphone that is the same as, or similar to, the microphone 160, and is integrated in the acoustic sensor 164 and (ii) a second microphone that is the same as, or similar to, the microphone 160, but is separate and distinct from the first microphone that is integrated in the acoustic sensor 164.

The RF transmitter 168 generates and/or emits radio waves having a predetermined frequency and/or a predetermined amplitude (e.g., within a high frequency band, within a low frequency band, long wave signals, short wave signals, etc.). The RF receiver 198 detects the reflections of the radio waves emitted from the RF transmitter 168, and this data can be analyzed by the control system 104 to determine a location of the user and/or one or more of the sleep-related parameters described herein. An RF receiver (either the RF receiver 198 and the RF transmitter 168 or another RF pair) can also be used for wireless communication between the control system 104, the respiratory therapy device 122, the one or more sensors 150, the user device 112, or any combination thereof. While the RF receiver 198 and RF transmitter 168 are shown as being separate and distinct elements in FIG. 1, in some implementations, the RF receiver 198 and RF transmitter 168 are combined as a part of an RF sensor 170 (e.g., a RADAR sensor). In some such implementations, the RF sensor 170 includes a control circuit. The format of the RF communication can be Wi-Fi, Bluetooth, or the like.

In some implementations, the RF sensor 170 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 170. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 172 outputs image data reproducible as one or more images (e.g., still images, video images, thermal images, or any combination thereof) that can be stored in the memory device 166. The image data from the camera 172 can be used by the control system 104 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 172 can be used to, for example, identify a location of the user, to determine chest movement of the user, to determine air flow of the mouth and/or nose of the user, to determine a time when the user enters the bed, and to determine a time when the user exits the bed. In some implementations, the camera 172 includes a wide angle lens or a fish eye lens.

The infrared (IR) sensor 150 outputs infrared image data reproducible as one or more infrared images (e.g., still images, video images, or both) that can be stored in the memory device 166. The infrared data from the IR sensor 150 can be used to determine one or more sleep-related parameters during a sleep session, including a temperature of the user and/or movement of the user. The IR sensor 150 can also be used in conjunction with the camera 172 when measuring the presence, location, and/or movement of the user. The IR sensor 150 can detect infrared light having a wavelength between about 700 nm and about 1 mm, for example, while the camera 172 can detect visible light having a wavelength between about 380 nm and about 740 nm.

The PPG sensor 176 outputs physiological data associated with the user that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 176 can be worn by the user, embedded in clothing and/or fabric that is worn by the user, embedded in and/or coupled to the user interface 132 and/or its associated headgear (e.g., straps, etc.), etc.

The ECG sensor 178 outputs physiological data associated with electrical activity of the heart of the user. In some implementations, the ECG sensor 178 includes one or more electrodes that are positioned on or around a portion of the user during the sleep session. The physiological data from the ECG sensor 178 can be used, for example, to determine one or more of the sleep-related parameters described herein.

The EEG sensor 180 outputs physiological data associated with electrical activity of the brain of the user. In some implementations, the EEG sensor 180 includes one or more electrodes that are positioned on or around the scalp of the user during the sleep session. The physiological data from the EEG sensor 180 can be used, for example, to determine a sleep state and/or a sleep stage of the user at any given time during the sleep session. In some implementations, the EEG sensor 180 can be integrated in the user interface 132 and/or the associated headgear (e.g., straps, etc.).

The capacitive sensor 182, the force sensor 184, and the strain gauge sensor 186 output data that can be stored in the memory device 166 and used/analyzed by the control system 104 to determine, for example, one or more of the sleep-related parameters described herein. The EMG sensor 188 outputs physiological data associated with electrical activity produced by one or more muscles. The oxygen sensor 194 outputs oxygen data indicative of an oxygen concentration of gas (e.g., in the conduit 140 or at the user interface 132). The oxygen sensor 194 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 190 can be used to detect the presence of an analyte in the exhaled breath of the user. The data output by the analyte sensor 190 can be stored in the memory device 166 and used by the control system 104 to determine the identity and concentration of any analytes in the breath of the user. In some implementations, the analyte sensor 190 is positioned near a mouth of the user to detect analytes in breath exhaled from the user's mouth. For example, when the user interface 132 is a facial mask (also known as a full face mask) that covers the nose and mouth of the user, the analyte sensor 190 can be positioned within the facial mask to monitor the user's mouth breathing. In other implementations, such as when the user interface 132 is a nasal mask or a nasal pillow mask, the analyte sensor 190 can be positioned near the nose of the user to detect analytes in breath exhaled through the user's nose. In still other implementations, the analyte sensor 190 can be positioned near the user's mouth when the user interface 132 is a nasal mask or a nasal pillow mask. In this implementation, the analyte sensor 190 can be used to detect whether any air is inadvertently leaking from the user's mouth and/or the user interface 132. In some implementations, the analyte sensor 190 is a volatile organic compound (VOC) sensor that can be used to detect carbon-based chemicals or compounds. In some implementations, the analyte sensor 190 can also be used to detect whether the user is breathing through their nose or mouth. For example, if the data output by an analyte sensor 190 positioned near the mouth of the user or within the facial mask (e.g., in implementations where the user interface 132 is a facial mask) detects the presence of an analyte, the control system 104 can use this data as an indication that the user is breathing through their mouth.

The moisture sensor 196 outputs data that can be stored in the memory device 166 and used by the control system 104. The moisture sensor 196 can be used to detect moisture in various areas surrounding the user (e.g., inside the conduit 140 or the user interface 132, near the user's face, near the connection between the conduit 140 and the user interface 132, near the connection between the conduit 140 and the respiratory therapy device 122, etc.). Thus, in some implementations, the moisture sensor 196 can be coupled to or integrated in the user interface 132 or in the conduit 140 to monitor the humidity of the pressurized air from the respiratory therapy device 122. In other implementations, the moisture sensor 196 is placed near any area where moisture levels need to be monitored. The moisture sensor 196 can also be used to monitor the humidity of the ambient environment surrounding the user, for example, the air inside the bedroom.

The Light Detection and Ranging (LiDAR) sensor 150 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 192 can measure and map an area extending 5 meters or more away from the sensor. The LiDAR data can be fused with point cloud data estimated by an electromagnetic RADAR sensor, for example. A LiDAR sensor 192 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 150 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 150 can be integrated in and/or coupled to any one or more of the components of the system 102, including the respiratory therapy device 122, the user interface 132, the conduit 140, the humidifier 144, the control system 104, the user device 112, the activity tracker 116, or any combination thereof. For example, the microphone 160 and the speaker 162 can be integrated in and/or coupled to the user device 112 and the pressure sensor 152 and/or flow rate sensor 154 are integrated in and/or coupled to the respiratory therapy device 122. In some implementations, at least one of the one or more sensors 150 is not coupled to the respiratory therapy device 122, the control system 104, or the user device 112, and is positioned generally adjacent to the user during the sleep session (e.g., positioned on or in contact with a portion of the user, worn by the user, coupled to or positioned on the nightstand, coupled to the mattress, coupled to the ceiling, etc.).

One or more of the respiratory therapy device 122, the user interface 132, the conduit 140, the display device 114, and the humidifier 144 can contain one or more sensors (e.g., a pressure sensor, a flow rate sensor, or more generally any of the other sensors 150 described herein). These one or more sensors can be used, for example, to measure the air pressure and/or flow rate of pressurized air supplied by the respiratory therapy device 122.

The data from the one or more sensors 150 can be analyzed (e.g., by the control system 104) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 150, or from other types of data.

The user device 112 can include a display device 114. The user device 112 can be, for example, a mobile device such as a smart phone, a tablet, a gaming console, a smart watch, a laptop, or the like. Alternatively, the user device 112 can be an external sensing system, a television (e.g., a smart television) or another smart home device (e.g., a smart speaker(s) such as Google Home™, Amazon Echo™, Alexa™, etc.). In some implementations, the user device is a wearable device (e.g., a smart watch). The display device 114 is generally used to display image(s) including still images, video images, or both. In some implementations, the display device 114 acts as a human-machine interface (HMI) that includes a graphic user interface (GUI) configured to display the image(s) and an input interface. The display device 114 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 112. In some implementations, one or more user devices can be used by and/or included in the system 102.

In some implementations, the system 102 also includes an activity tracker 116. The activity tracker 116 is generally used to aid in generating physiological data associated with the user. The activity tracker 116 can include one or more of the sensors 150 described herein, such as, for example, the motion sensor 158 (e.g., one or more accelerometers and/or gyroscopes), the PPG sensor 176, and/or the ECG sensor 178. The physiological data from the activity tracker 116 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 116 is coupled (e.g., electronically or physically) to the user device 112.

In some implementations, the activity tracker 116 is a wearable device that can be worn by the user, such as a smartwatch, a wristband, a ring, or a patch. For example, in some cases an activity tracker 116 is worn on a wrist of the user. The activity tracker 116 can also be coupled to or integrated a garment or clothing that is worn by the user. Alternatively still, the activity tracker 116 can also be coupled to or integrated in (e.g., within the same housing) the user device 112. More generally, the activity tracker 116 can be communicatively coupled with, or physically integrated in (e.g., within a housing), the control system 104, the memory device 166, the respiratory therapy system 120, and/or the user device 112.

In some implementations, the system 102 also includes a blood pressure device 118. The blood pressure device 118 is generally used to aid in generating cardiovascular data for determining one or more blood pressure measurements associated with the user. The blood pressure device 118 can include at least one of the one or more sensors 150 to measure, for example, a systolic blood pressure component and/or a diastolic blood pressure component.

In some implementations, the blood pressure device 118 is a sphygmomanometer including an inflatable cuff that can be worn by the user and a pressure sensor (e.g., the pressure sensor 152 described herein). For example, in some cases a blood pressure device 118 can be worn on an upper arm of the user. In such implementations where the blood pressure device 118 is a sphygmomanometer, the blood pressure device 118 also includes a pump (e.g., a manually operated bulb) for inflating the cuff. In some implementations, the blood pressure device 118 is coupled to the respiratory therapy device 122 of the respiratory therapy system 120, which in turn delivers pressurized air to inflate the cuff. More generally, the blood pressure device 118 can be communicatively coupled with, and/or physically integrated in (e.g., within a housing), the control system 104, the memory device 166, the respiratory therapy system 120, the user device 112, and/or the activity tracker 116.

In other implementations, the blood pressure device 118 is an ambulatory blood pressure monitor communicatively coupled to the respiratory therapy system 120. An ambulatory blood pressure monitor includes a portable recording device attached to a belt or strap worn by the user and an inflatable cuff attached to the portable recording device and worn around an arm of the user. The ambulatory blood pressure monitor is configured to measure blood pressure between about every fifteen minutes to about thirty minutes over a 24-hour or a 48-hour period. The ambulatory blood pressure monitor may measure heart rate of the user at the same time. These multiple readings are averaged over the 24-hour period. The ambulatory blood pressure monitor determines any changes in the measured blood pressure and heart rate of the user, as well as any distribution and/or trending patterns of the blood pressure and heart rate data during a sleeping period and an awakened period of the user. The measured data and statistics may then be communicated to the respiratory therapy system 120.

The blood pressure device 118 maybe positioned external to the respiratory therapy system 120, coupled directly or indirectly to the user interface 132, coupled directly or indirectly to a headgear associated with the user interface 132, or inflatably coupled to or about a portion of the user. The blood pressure device 118 is generally used to aid in generating physiological data for determining one or more blood pressure measurements associated with a user, for example, a systolic blood pressure component and/or a diastolic blood pressure component. In some implementations, the blood pressure device 118 is a sphygmomanometer including an inflatable cuff that can be worn by a user and a pressure sensor (e.g., the pressure sensor 152 described herein).

In some implementations, the blood pressure device 118 is an invasive device which can continuously monitor arterial blood pressure of the user and take an arterial blood sample on demand for analyzing gas of the arterial blood. In some other implementations, the blood pressure device 118 is a continuous blood pressure monitor, using a radio frequency sensor and capable of measuring blood pressure of the user once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra-wideband ranges (which may include spreading, PRN codes or impulse systems).

While the control system 104 and the memory device 166 are described and shown in FIG. 1 as being a separate and distinct component of the system 102, in some implementations, the control system 104 and/or the memory device 166 are integrated in the user device 112 and/or the respiratory therapy device 122. Alternatively, in some implementations, the control system 104 or a portion thereof (e.g., the processor 108) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 102 is shown as including all of the components described above, more or fewer components can be included in a system according to implementations of the present disclosure. For example, a first alternative system includes the control system 104, the memory device 166, and at least one of the one or more sensors 150 and does not include the respiratory therapy system 120. As another example, a second alternative system includes the control system 104, the memory device 166, at least one of the one or more sensors 150, and the user device 112. As yet another example, a third alternative system includes the control system 104, the memory device 166, the respiratory therapy system 120, at least one of the one or more sensors 150, and the user device 112. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes up and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 114 of the user device 112 to manually initiate or terminate the sleep session.

Generally, the sleep session includes any point in time after the user has laid or sat down in the bed (or another area or object on which they intend to sleep), and has turned on the respiratory therapy device 122 and donned the user interface 132. The sleep session can thus include time periods (i) when the user is using the respiratory therapy system 120, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep.

The sleep session is generally defined as ending once the user removes the user interface 132, turns off the respiratory therapy device 122, and gets out of bed. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user, ending when the respiratory therapy device 122 stops supplying the pressurized air to the airway of the user, and including some or all of the time points in between, when the user is asleep or awake.

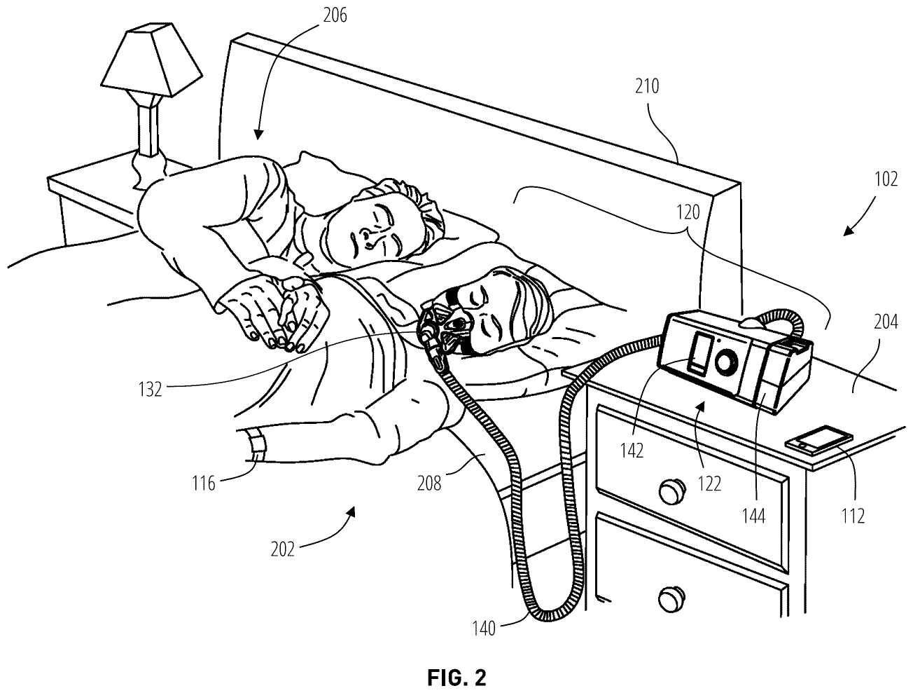

FIG. 2 is a perspective view of at least a portion of a system such as the system 102 of FIG. 1, a user 202, and a bed partner 206, according to certain aspects of the present disclosure. System 102 is depicted in FIG. 2 with certain components, such as a respiratory therapy system 120, a user device 112, an activity tracker 116, and the like. In some cases, such as described in further detail herein with reference to FIG. 1, system 102 can include other arrangements of components.

As shown in FIG. 2, the respiratory therapy system 120 can be used to treat a user 202. In this example, the user 202 of the respiratory therapy system 120 and a bed partner 206 are located in a bed 210 and are laying on a mattress 208. User 202 and bed partner 206 can be the user and bed partner as described in further detail with reference to FIG. 1.

The respiratory therapy system 120 can include various components, such as a respiratory therapy device 122, a user interface 132, and a conduit 140 fluidly (e.g., pneumatically) coupling the respiratory therapy device 122 and the user interface 132. The respiratory therapy device 122 can include various components, such as a humidifier 144 and display device 142.

The user interface 132 can be worn by the user 202 during a sleep session. The respiratory therapy system 120 generally aids in increasing the air pressure in the throat of the user 202 to aid in preventing the airway from closing and/or narrowing during sleep. The respiratory therapy device 122 can be positioned on a nightstand 204 that is directly adjacent to the bed 210, or more generally, on any surface or structure that is generally adjacent to the bed 210 and/or the user 202.

As shown in FIG. 2, in some implementations, the user interface 132 is a facial mask (e.g., a full face mask) that covers at least a portion of the nose and mouth of the user 202. Alternatively, the user interface 132 can be a nasal mask that provides air to the nose of the user or a nasal pillow mask that delivers air directly to the nostrils of the user 202. In other implementations, the user interface 132 includes a mouthpiece (e.g., a night guard mouthpiece molded to conform to the teeth of the user, a mandibular repositioning device, etc.).

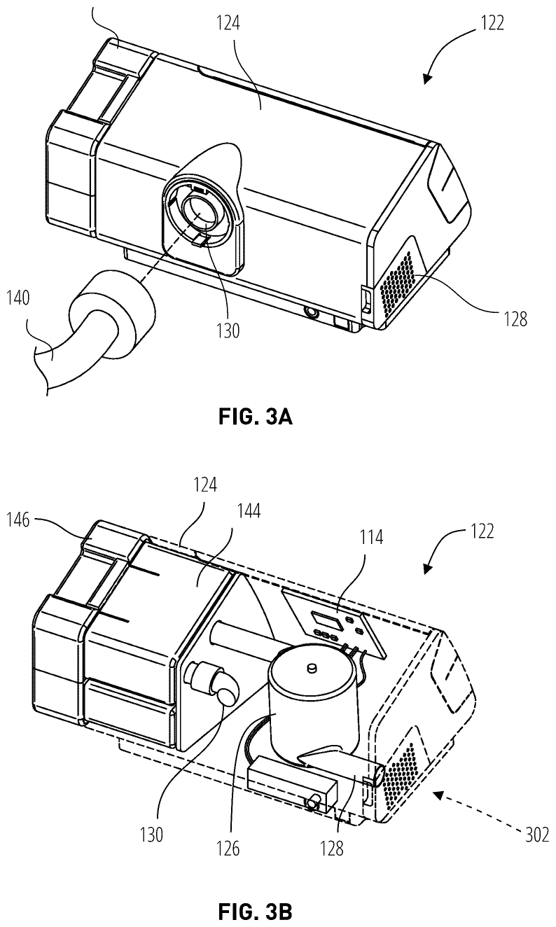

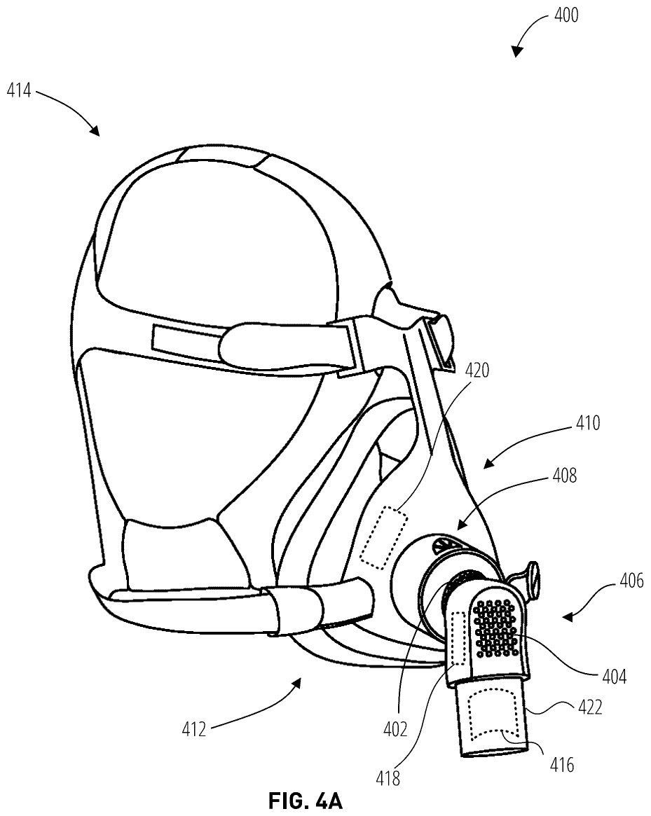

FIG. 3A is a perspective view of a respiratory therapy device 122 of the system 102 of FIG. 1, according to some implementations of the present disclosure. The respiratory therapy device 122 as depicted in FIG. 3A can be the same as the respiratory therapy device 122 as depicted in FIG. 2, except rotated by 180° such that the air inlet 128 faces towards the bottom left of FIG. 3A as opposed to the top right of FIG. 2.

The respiratory therapy device 122 includes an air inlet 128 for receiving ambient air and an air outlet 130 for delivering pressurized air. In some implementations, the air inlet 128 and/or the air outlet 130 include a cover that is moveable between a closed position and an open position (e.g., to prevent or inhibit air from flowing through the air inlet 128 or the air outlet 130). As described in further detail herein, the conduit 140 is coupled to the air outlet 130 of the respiratory therapy device 122. In some cases, the respiratory therapy device 122 can include a reservoir 146 for storing water for humidification of the air being pressurized to the air outlet 130. A housing 124 can protect the interior components of the respiratory therapy device 122.

FIG. 3B is a perspective view of the respiratory therapy device 122 of FIG. 3A illustrating an interior of a housing 124, according to some implementations of the present disclosure. For illustrative purposes, housing 124 is depicted as transparent.

The blower motor 126 is at least partially disposed or integrated within the housing 124. The blower motor 126 draws air from outside the housing 124 (e.g., atmosphere) via the air inlet 128 and causes pressurized air to flow through the humidifier 144, and through the air outlet 130. For example, air may flow from the air inlet 128 through the blower motor 126, and then through the humidifier 144 before exiting the respiratory therapy device 122 via the air outlet 130. In some cases, the housing 124 can include a vent 302 to allow air to pass through the housing 124 to the air inlet 128. When the respiratory therapy device 122 includes a humidifier 144, a reservoir 146 can store water for use by the humidifier 144.

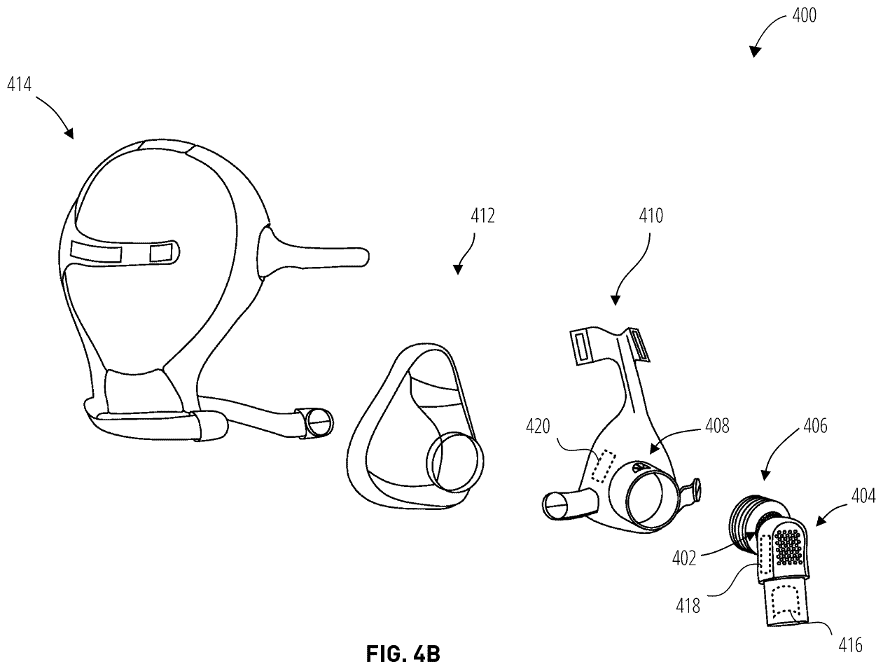

FIG. 4A is a perspective view of a user interface 400, according to some implementations of the present disclosure. User interface 400 can be the same as, or similar to, the user interface 132 of FIG. 2.

The user interface 400 generally includes a cushion 412 and a frame 410 that define a volume of space around the mouth and/or nose of the user. When in use, the volume of space receives pressurized air for passage into the user's airways. In some implementations, the cushion 412 and frame 410 of the user interface 400 form a unitary component of the user interface. The user interface 400 can also include a headgear 414, which generally includes a strap assembly and optionally a connector 406. The headgear 414 is configured to be positioned generally about at least a portion of a user's head when the user wears the user interface 400. The headgear 414 can be coupled to the frame 410 and positioned on the user's head such that the user's head is positioned between the headgear 414 and the frame 410. The cushion 412 is positioned between the user's face and the frame 410 to form a seal on the user's face. The optional connector 406 is configured to couple to the frame 410 and/or cushion 412 at one end and to a conduit of a respiratory therapy device (e.g., conduit 140 of FIG. 1). The pressurized air can flow directly from the conduit of the respiratory therapy system into the volume of space defined by the cushion 412 (or cushion 412 and frame 410) of the user interface 400 through the connector 406). From the user interface 400, the pressurized air reaches the user's airway through the user's mouth, nose, or both. Alternatively, where the user interface 400 does not include the connector 406, the conduit of the respiratory therapy system can connect directly to the cushion 412 and/or the frame 410.

In some implementations, the connector 406 may include one or more vents 404 (e.g., a plurality of vents) located on the main body of the connector 406 itself and/or one or a plurality of vents 402 (“diffuser vents”) in proximity to the frame 410, for permitting the escape of carbon dioxide (CO₂) and other gases exhaled by the user. In some implementations, one or a plurality of vents, such as vents 404 and/or 402 may be located in the user interface 400, such as in frame 410, and/or in the conduit (e.g., conduit 140 of FIG. 1). In some implementations, the frame 410 includes at least one anti-asphyxia valve 408 (AAV), which allows CO₂ and other gases exhaled by the user to escape in the event that the vents (e.g., the vents 404 or 402) fail when the respiratory therapy device is active. In general, AAVs (e.g., the AAV 408) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 402, other masks might have only the plurality of vents 404 on the connector itself).

According to certain aspects of the present disclosure, one or more motion sensors 416, 418, 420 can be coupled, directly or indirectly, to the user interface 400. For example, a motion sensor 416 may be indirectly coupled to the user interface 400 by being coupled to a conduit 422, which is itself coupled to a frame 410 of the user interface 400 via a connector 406. As another example, a motion sensor 418 can be coupled to a connector 406 of the user interface 400. As another example, a motion sensor 420 can be coupled to a frame 410 of the user interface 400. Motion sensors can be coupled to a user interface 400 in other ways and at other locations.

In some cases, a motion sensor can wirelessly communicate with a receiving device (e.g., a user device 112 or other computing device) to convey collected motion data for analysis as disclosed in further detail herein. In some cases, however, a motion sensor can be communicatively coupled with a receiving device via a wired connection.

FIG. 4B is an exploded view of the user interface 400 of FIG. 4A, according to some implementations of the present disclosure. As depicted in FIG. 4B, the user interface 400 comprises headgear 414 that can couple to a frame 410 to secure a cushion 412 in place. A connector 406 can couple to the frame 410, and/or cushion 412, to deliver air from the conduit to the user interface. The connector 406 can include vents 404 and/or 402. The frame 410 can include an anti-asphyxia valve 408.

According to certain aspects of the present disclosure, one or more motion sensors 416, 418, 420 can be coupled, directly or indirectly, to the user interface 400.

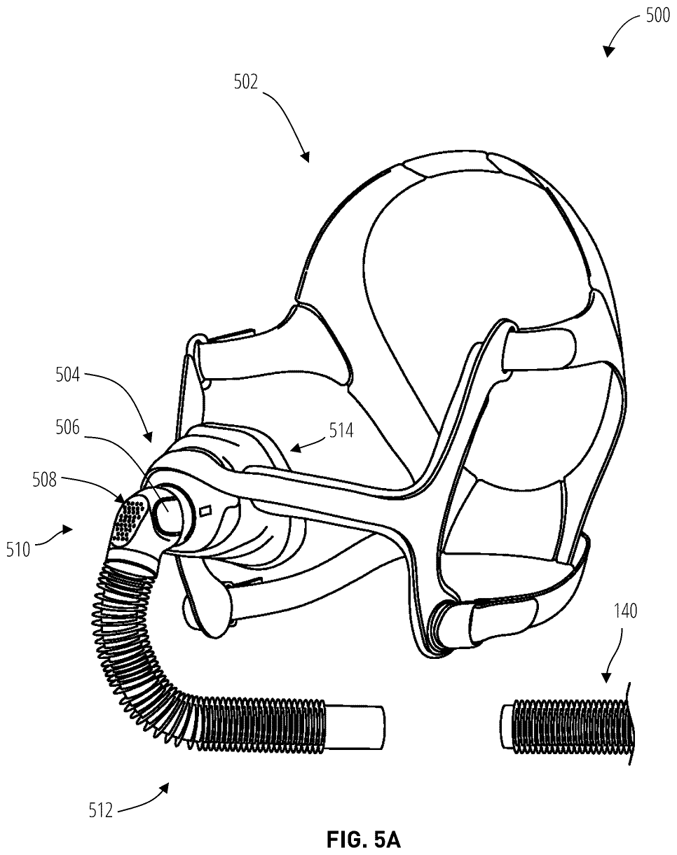

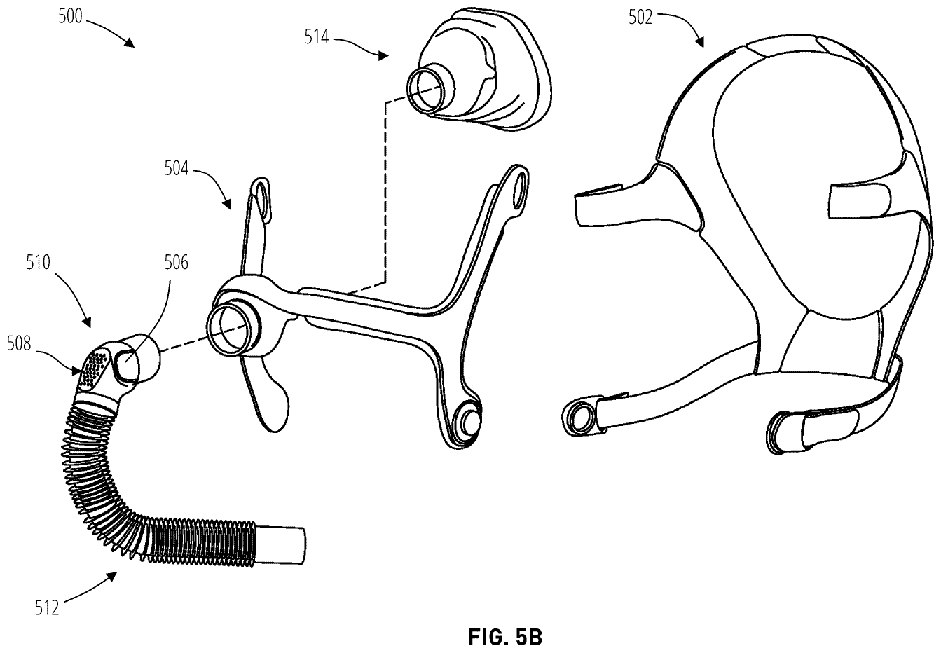

FIG. 5A is a perspective view of a user interface 500, according to some implementations of the present disclosure. User interface 500 can be the same as, or similar to, the user interface 132 of FIG. 1.

The user interface 500 differs from some other user interfaces in that the user interface 500 is an indirect user interface, whereas some other user interfaces are direct user interfaces. The user interface 500 includes a headgear 502 (e.g., as a strap assembly), a cushion 514, a frame 504, a connector 510, and a user interface conduit 512 (often referred to as a minitube or a flexitube). The user interface 500 is an indirectly connected user interface because pressurized air is delivered from the conduit 140 of the respiratory therapy system to the cushion 514 and/or frame 504 through the user interface conduit 512, rather than directly from the conduit 140 of the respiratory therapy system.

In some implementations, the cushion 514 and frame 504 form a unitary component of the user interface 500. Generally, the user interface conduit 512 is more flexible than the conduit 140 of the respiratory therapy system described above and/or has a diameter smaller than the diameter of the than the than the conduit 140. The user interface conduit 512 is typically shorter that conduit 140. Similar to the headgear of other user interfaces, the headgear 502 of user interface 500 is configured to be positioned generally about at least a portion of a user's head when the user wears the user interface 500. The headgear 502 can be coupled to the frame 504 and positioned on the user's head such that the user's head is positioned between the headgear 502 and the frame 504. The cushion 514 is positioned between the user's face and the frame 504 to form a seal on the user's face. The connector 510 is configured to couple to the frame 504 and/or cushion 514 at one end and to the user interface conduit 512 of the user interface 500 at the other end. In other implementations, the user interface conduit 512 may connect directly to frame 504 and/or cushion 514. The user interface conduit 512, at the opposite end relative to the frame 504 and cushion 514, is configured to connect to the conduit 140. The pressurized air can flow from the conduit 140 of the respiratory therapy system, through the user interface conduit 512, and the connector 510, and into a volume of space define by the cushion 514 (or cushion 514 and frame 504) of the user interface 500 against a user's face. From the volume of space, the pressurized air reaches the user's airway through the user's mouth, nose, or both.

In some implementations, the connector 510 includes a plurality of vents 508 for permitting the escape of carbon dioxide (CO₂) and other gases exhaled by the user when the respiratory therapy device is active. In such implementations, each of the plurality of vents 508 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 508 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device, and passes through the conduit and to the user interface 500.

In some implementations, the connector 510 optionally includes at least one valve 506 for permitting the escape of CO₂ and other gases exhaled by the user when the respiratory therapy device is inactive. In some implementations, the valve 506 (an example of an anti-asphyxia valve) includes a silicone (or other suitable material) flap that is a failsafe component, which allows CO₂ and other gases exhaled by the user to escape in the event that the vents 508 fail when the respiratory therapy device is active. In such implementations, when the silicone flap is open, the valve opening is much greater than each vent opening, and therefore less likely to be blocked by occlusion materials.

FIG. 5B is an exploded view of the user interface 500 of FIG. 5A, according to some implementations of the present disclosure. As depicted in FIG. 5B, the user interface 500 comprises a headgear 502 that can couple to frame 504. The frame 504 can have an opening through which a portion of a cushion 514 can be inserted. The headgear 502 and frame 504 can thus secure the cushion 514 against the face of a user when the user interface 500 is worn by a user. A connector 510 can couple a user interface conduit 512 to the cushion 514. The connector 510 can include one or more vents 508 and optionally a valve 506.

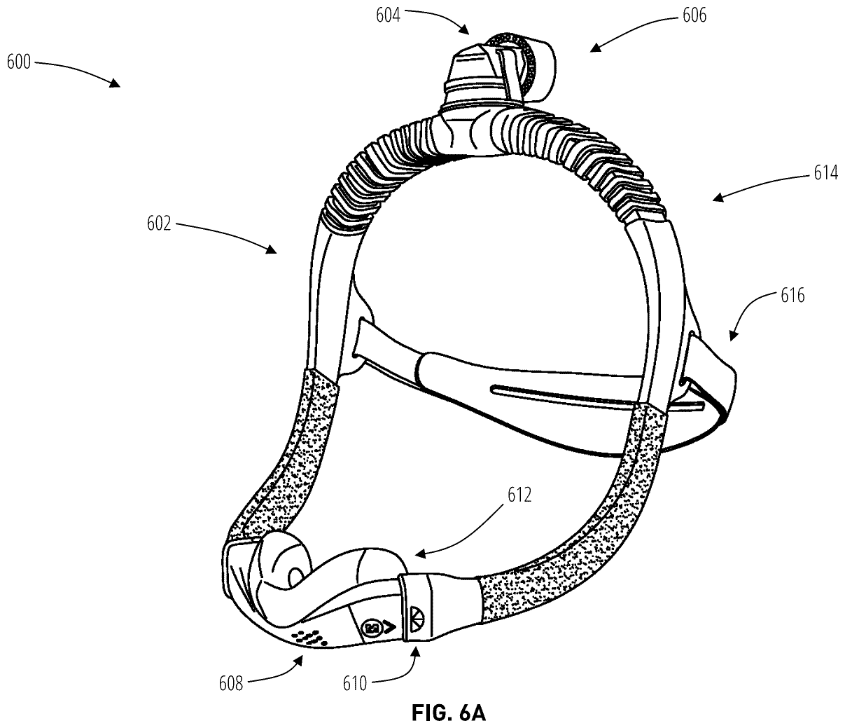

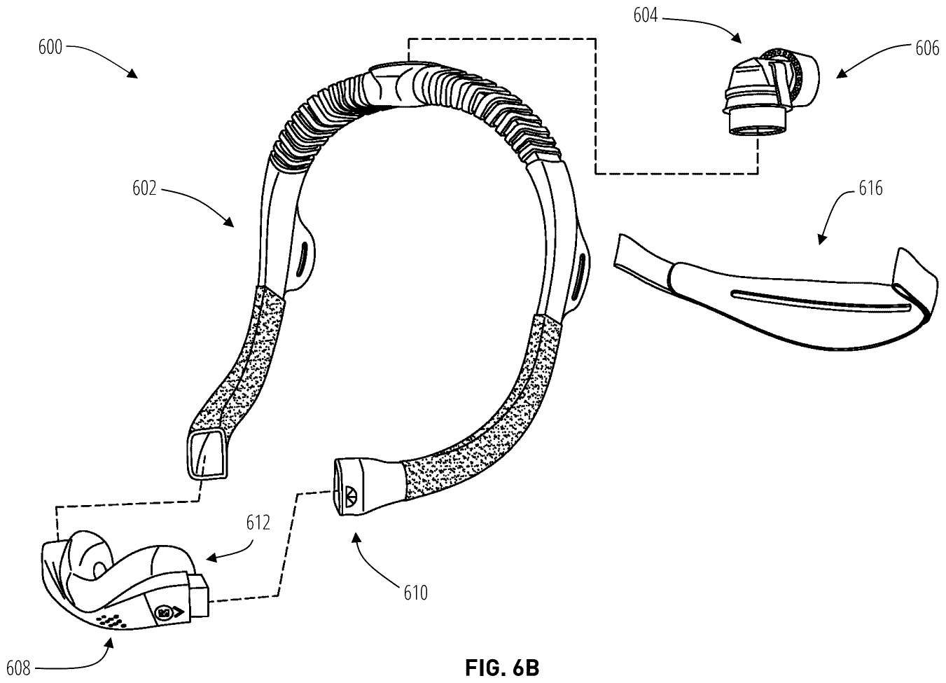

FIG. 6A is a perspective view of a user interface 600, according to some implementations of the present disclosure. User interface 600 can be the same as, or similar to, the user interface 132 of FIG. 1.

The user interface 600 is similar to other indirect user interfaces. The indirect headgear user interface 600 includes headgear 614, a cushion 612, and a connector 606. The headgear 614 includes headgear strap 616 and a headgear conduit 602. Similar to some other user interfaces, the headgear 614 is configured to be positioned generally about at least a portion of a user's head when the user wears the user interface 600. The headgear 614 includes a headgear strap 616 that can be coupled to the headgear conduit 602 and positioned on the user's head such that the user's head is positioned between the headgear strap 616 and the headgear conduit 602. The cushion 612 is positioned between the user's face and the headgear conduit 602 to form a seal on the user's face.

The connector 606 is configured to couple to the headgear 614 at one end and a conduit (e.g., conduit 140 of FIG. 1) of the respiratory therapy system at the other end. In other implementations, the connector 606 is not included and the headgear 614 can alternatively connect directly to conduit of the respiratory therapy system. The headgear conduit 602 can be configured to deliver pressurized air from the conduit of the respiratory therapy system to the cushion 612, or more specifically, to the volume of space around the mouth and/or nose of the user and enclosed by the user cushion. The headgear conduit 602 is hollow to provide a passageway for the pressurized air. Both sides of the headgear conduit 602 can be hollow to provide two passageways for the pressurized air. Alternatively, only one side of the headgear conduit 602 can be hollow to provide a single passageway. In the implementation illustrated in FIG. 6A, headgear conduit 602 comprises two passageways which, in use, are positioned at either side of a user's head/face. Alternatively, only one passageway of the headgear conduit 602 can be hollow to provide a single passageway. The pressurized air can flow from the conduit of the respiratory therapy system, through the connector 606 and the headgear conduit 602, and into the volume of space between the cushion 612 and the user's face. From the volume of space between the cushion 612 and the user's face, the pressurized air reaches the user's airway through the user's mouth, nose, or both.

In some implementations, the cushion 612 includes a plurality of vents 608 on the cushion 612 itself. Additionally or alternatively, in some implementations, the connector 606 includes a plurality of vent 604 (“diffuser vents”) in proximity to the headgear 614, for permitting the escape of carbon dioxide (CO₂) and other gases exhaled by the user when the respiratory therapy device is active. In some implementations, the headgear 614 may include at least one plus anti-asphyxia valve 610 in proximity to the cushion 612, which allows CO₂ and other gases exhaled by the user to escape in the event that the vents (e.g., the vent 604 or 608) fail when the respiratory therapy device is active.

FIG. 6B is an exploded view of the user interface 600 of FIG. 6A, according to some implementations of the present disclosure. The user interface 600 comprises a headgear conduit 602 couplable to a headgear strap 616, a connector 606, and a cushion 612. The headgear conduit 602 can convey air from a conduit (e.g., conduit 140 of FIG. 1), via the connector 606, to the cushion 612. The cushion 612 can include a vent 608 and/or the connector 606 can include a vent 604. In some cases, the headgear conduit 602 can include an anti-asphyxia valve 610.

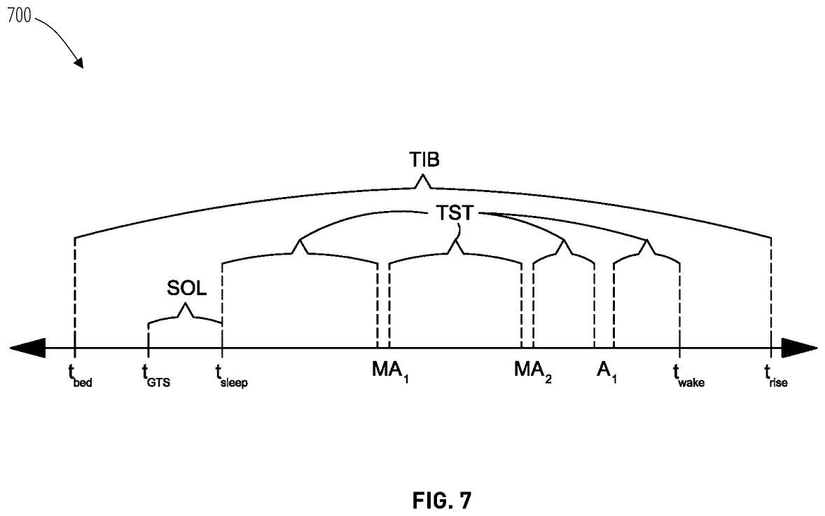

FIG. 7 illustrates an exemplary timeline 700 for a sleep session, according to some implementations of the present disclosure. Referring to the timeline 700, the enter bed time t_{bed} is associated with the time that the user initially enters the bed prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using a user device (e.g., user device 112 of FIG. 1), etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one or more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 700, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (teed) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

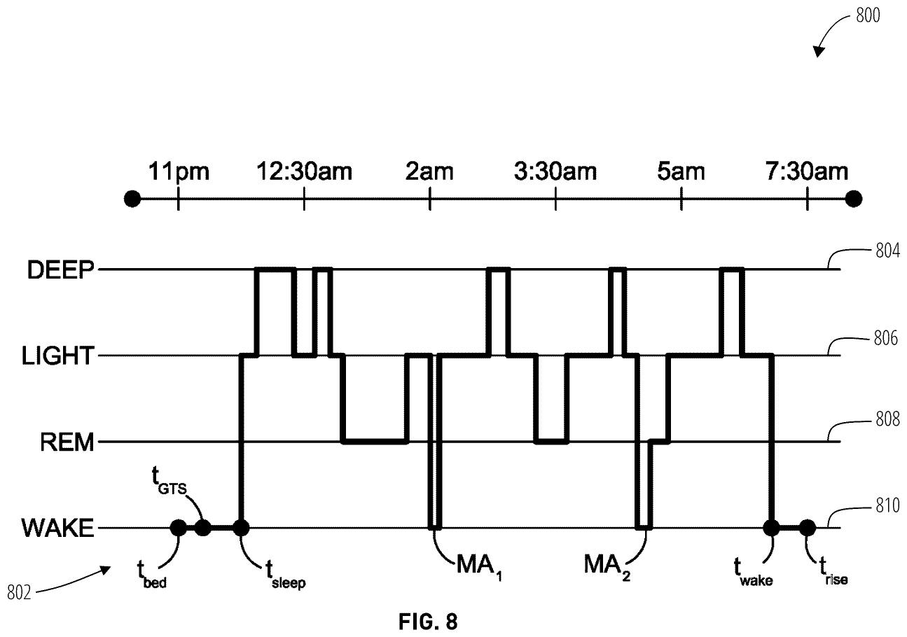

FIG. 8 illustrates an exemplary hypnogram 800 associated with the sleep session of FIG. 7, according to some implementations of the present disclosure. The exemplary hypnogram 800 corresponds to the timeline 700 of FIG. 7, according to some implementations, is illustrated. As shown, the hypnogram 800 includes a sleep-wake signal 802, a wakefulness stage axis 810, a REM stage axis 808, a light sleep stage axis 806, and a deep sleep stage axis 804. The intersection between the sleep-wake signal 802 and one of the axes 804, 806, 808, 810 is indicative of the sleep stage at any given time during the sleep session.

The sleep-wake signal 802 can be generated based on physiological data associated with the user (e.g., generated by one or more of the sensors (e.g., sensors 150 of FIG. 1) described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 800 is shown in FIG. 8 as including the light sleep stage axis 806 and the deep sleep stage axis 804, in some implementations, the hypnogram 800 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device (e.g., memory device 166 of FIG. 1).

The hypnogram 800 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 7), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.).

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 7), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake} Ae), the rising time (tᵣᵢₛₑ), or any combination thereof based at least in part on the sleep-wake signal of a hypnogram.

In other implementations, one or more of the sensors (e.g., sensors 150 of FIG. 1) can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by a motion sensor (e.g., motion sensor 158 of FIG. 1), a microphone (e.g., microphone 160 of FIG. 1), a camera (e.g., camera 172 of FIG. 1), or any combination thereof. The go-to-sleep time can be determined based on, for example, data from a motion sensor (e.g., motion sensor 158 of FIG. 1) (e.g., data indicative of no movement by the user), data from a camera (e.g., camera 172 of FIG. 1) (e.g., data indicative of no movement by the user and/or that the user has turned off the lights) data from a microphone (e.g., microphone 160 of FIG. 1) (e.g., data indicative of the user turning off a TV), data from a user device (e.g., user device 112 of FIG. 1) (e.g., data indicative of the user no longer using the user device), data from a pressure sensor (e.g., pressure sensor 152 of FIG. 1) and/or a flow rate sensor (e.g., flow rate sensor 154 of FIG. 1) (e.g., data indicative of the user turning on the respiratory therapy device (e.g., respiratory therapy device 122 of FIG. 1), data indicative of the user donning a user interface (e.g., user interface 132 of FIG. 1), etc.), or any combination thereof.

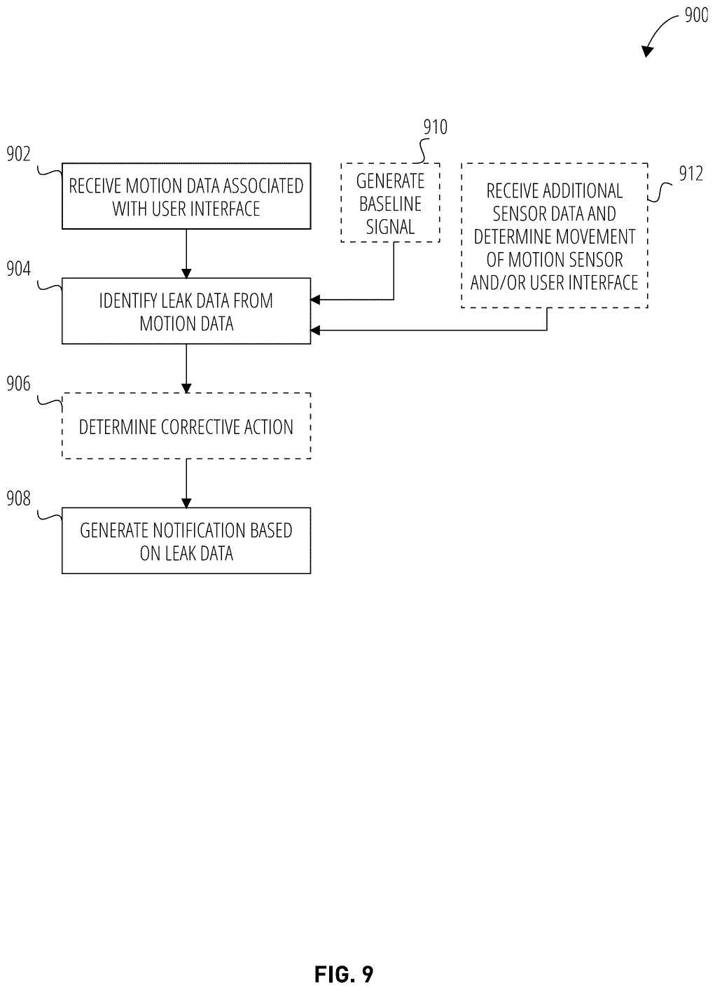

FIG. 9 is a flow chart depicting a process for analyzing user interface leak, according to certain aspects of the present disclosure. Process 900 can be performed using any suitable system, such as system 102 of FIG. 1.

At block 902, motion data associated with the user interface can be received. Motion data can comprise data indicative of movement of the user interface, such as linear acceleration (e.g., as measured by an accelerometer) or rotational velocity (e.g., as measured by a gyroscope). The motion data associated with the user interface can be acquired using one or more sensors coupled to the user interface. The one or more sensors can be coupled directly (e.g., an accelerometer coupled to a frame and/or cushion of a user interface) or indirectly (e.g., an accelerometer coupled to a conduit that is coupled a user interface, such as to a component of the user interface, for example, a frame of the user interface, optionally via a connector) to the user interface. As such, the one or more sensors can be coupled directly to the user interface, and can be integrated on or in (partially or wholly) the user interface. Alternatively, the one or more sensors can be coupled indirectly to the user interface, and can be integrated on or in (partially or wholly) the conduit and/or the connector coupled the user interface.

Motion data can include one or more streams of data, such as i) an x-axis linear acceleration data stream; ii) a y-axis linear acceleration data stream; iii) a z-axis linear acceleration data stream; iv) an x-axis rotational velocity data stream; v) a y-axis rotational velocity data stream; vi) a z-axis rotational velocity data stream; or vii) and combination of i-vi. As used herein, while the user is wearing the user interface and sitting in an upright position, an x-axis can be oriented horizontally across the user's face (e.g., in a left-right direction, such as parallel to a line from one eye to another eye, such as from the user's perspective where movement in a direction towards the user's right ear is positive), a z-axis can be oriented into and out of the user's face (e.g., a front-back direction, such as parallel to a line between the user's nose and back of the head, such as from the user's perspective where movement in a direction from the user's head and out through the nose is positive), and a y-axis can be oriented vertically along a user's face (e.g., in an up-down direction, such as parallel a line from the user's neck to a top of the user's head, such as from the user's perspective where movement in a direction from the user's head up and out through the top of the user's head is positive). In some cases, other orientations can be used for axes. Linear acceleration can be defined as acceleration along an axis, whereas rotational velocity can be defined as rotational velocity about an axis.

As described in various examples herein, motion data can be obtained via a sensor (e.g., accelerometer) coupled to the user interface, although that need not always be the case. In some cases, motion data associated with the user interface can be obtained remotely, such as via remote measurement of the position and/or orientation of the user interface via one or more remote sensors. For example, an RF sensor or a LiDAR sensor can be used to identify changes in position and/or orientation of the user interface with respect to the sensor, which can be used as motion data. For example, a sensor incorporated into a device sitting on a nightstand can be used to measure or estimate changes in the position and/or orientation of the user interface with respect to the nightstand.

At block 904, leak data can be identified from the motion data. Identifying leak data at block 904 can include determining the presence of, intensity of (e.g., volume of air leak per unit time), and/or location of an unintentional leak. As used herein, determination of a location of an unintentional leak can include determining a relative location of the unintentional leak with respect to the user interface. The location can be a specific location on the user interface (e.g., an exact point where the unintentional leak occurred) or a general location (e.g., a general indication that the unintentional leak is occurring in a certain region of the user interface where it contacts the user's face, such as the top-left or top-right quadrant of the user interface). In some cases, identifying leak data at block 904 can include identifying a strap or other securement feature of the user interface, the suboptimal positioning or tightening of which may be causing the unintentional leak. For example, an unintentional leak may be identified as a “top-left strap” leak if adjustment of the top-left strap would reduce, minimize, or eliminate the leak.

In some cases, identifying leak data from the motion data can include using the motion data to classify an unintentional leak. Classifying an unintentional leak can include classifying the unintentional leak i) based on the intensity of the unintentional leak; ii) based on the location of the unintentional leak; iii) based on a component of the user interface (e.g., cushion) where the unintentional leak is occurring; iv) based on a component of the user interface (e.g., strap) that can be adjusted to reduce the unintentional leak; or v) any combination of i-iv. Other classification schemes can be used.

In some cases, analyzing the motion data to identify leak data can include identifying body position data indicative of a body position of the user based at least in part on the motion data. For example, it has been determined that the position of the user's body (e.g., supine or on a particular side) can be indicative of the likely cause of the unintentional leak. For example, when in a supine position, there is a significantly higher likelihood that an unintentional leak is pressure related than the unintentional leak being movement related, spontaneous, or part of a leak chain. Likewise, when in a lateral position (e.g., on one side or another), there is a greater likelihood that an unintentional leak is movement related than the unintentional leak being pressure related, spontaneous, or part of a leak chain. Leveraging body position data, the system can determine a likely cause of the unintentional leak, which can be further used to provide informative notifications and/or to help determine any corrective actions that can be taken to reduce the unintentional leak.

Additionally, in some cases, changes in body position can be used to adjust the motion data to compensate for the motion of changing body position. For example, determined rotation of the body about an axis (e.g., a y-axis) can be used to offset rotational velocity data (e.g., rotational velocity data) about that same axis, thus resulting in corrected rotational velocity data of the user interface with respect to the user.

In some cases, identifying leak data at block 904 can include comparing motion data (e.g., processed motion data, such as features from motion data) to a baseline signal. A baseline signal can be generated at block 910, which can be separate from or part of block 904. Generating the baseline signal at block 910 can include analyzing motion data to determine that the user is likely not experiencing an unintentional leak or that any experienced unintentional leak is likely low (e.g., negligible). The motion data analyzed to generate the baseline signal can be motion data from the same sleep session during which an unintentional leak is detected at block 904 (e.g., motion data from soon after the user started respiratory therapy, as it may be expected that the user interface would not have significant unintentional leaks at that time), although that need not always be the case. In some cases, the motion data analyzed to generate the baseline signal can be motion data from a prior sleep session or prior fitting session (such as a therapy titration session), where it is known or estimated that no or minimal unintentional leak occurred. In some cases, the motion data analyzed to generate the baseline signal can be based on an alternative leak signal, such as one generated without the use of the motion data from block 902. The motion data analyzed to generate the baseline signal can thus be motion data associated with instances where the alternative leak signal indicates the occurrence of no or minimal unintentional leak. For example, traditional methods to measure unintentional leak may make use of the pressure versus flow curve (PQ curve) as detected by the respiratory therapy device to generate an estimated leak signal (e.g., a signal indicative of the estimated presence of or intensity of an unintentional leak). In such an example, the motion data analyzed to generate the baseline signal can be motion data associated with the estimated leak signal being at or below a threshold value (e.g., the estimated leak signal indicating there is a low or negligible leak).

In some cases, comparing motion data to a baseline signal can include determining a difference between the motion data and the baseline signal, in which case a leak may be identified and/or classified when the difference exceeds a threshold value or falls within a threshold range.

In some cases, identifying leak data from motion data can include applying the motion data to a machine learning algorithm. Such a machine learning algorithm can be trained using training data comprising motion data measurements acquired as mask pressure is manipulated between high and low values, thus causing any unintentional leaks to leak with higher and lower intensity and/or at different locations. Differential motion data can be defined as differences between the motion data while the unintentional leak is high and the motion data while the unintentional leak is low or not present. In some cases, training data can further include user-provided leak information, such as user-reported indications of the presence, intensity, and/or location of an unintentional leak. In some cases, training data can further include user-provided corrective action information. User-provided corrective action information can include information about what corrective actions were taken to address any unintentional leaks that occurred while the motion data was being acquired (e.g., the bottom-right strap was pulled tighter), and optionally the efficacy of the corrective action (e.g., pulling the bottom-right strap tighter seemed to improve the unintentional leak).

Identifying leak data at block 904 can include processing motion data (e.g., raw motion data or differential motion data) to obtain time-domain motion data (e.g., time-domain acceleration data and/or time-domain rotational velocity data) and/or frequency-domain motion data (e.g., frequency-domain acceleration data and/or frequency-domain rotational velocity data). Unintentional leaks can be identified based on the time-domain motion data and/or frequency-domain motion data. In an example, one or more features can be extracted from the time-domain motion data and/or frequency-domain motion data. In some cases, time-domain motion data and/or frequency-domain motion data from raw motion data can be compared with time-domain motion data and/or frequency-domain motion data from baseline motion data (e.g., motion data collected when the unintentional leak is low or when no unintentional leak is present) to obtain a differential value (e.g., a frequency-domain motion deviation feature) that can be used to classify an unintentional leak.

Any number of features can be determined from motion data, such as i) an x-axis linear acceleration orientation displacement feature; ii) a y-axis linear acceleration orientation displacement feature; iii) a z-axis linear acceleration orientation displacement feature; iv) an x-axis rotational velocity displacement feature; v) a y-axis rotational velocity displacement feature; vi) a z-axis rotational velocity displacement feature; vii) an x-axis linear acceleration frequency-domain deviation feature; viii) a y-axis linear acceleration frequency-domain deviation feature; ix) a z-axis linear acceleration frequency-domain deviation feature; x) an x-axis rotational velocity frequency-domain deviation feature; xi) a y-axis rotational velocity frequency-domain deviation feature; xii) a z-axis rotational velocity frequency-domain deviation feature; or xiii) any combination of i-xii.

An orientation displacement feature (e.g., a user interface orientation displacement feature, such as items i-iii immediately above) can include a value indicative of a shift in the orientation of the user interface (e.g., as measured from an averaged accelerometer reading, such as an averaged DC accelerometer reading) between the received motion data and a baseline (e.g., motion data acquired while no unintentional leak is occurring or while any unintentional leaks are only minimally occurring). An orientation displacement feature can be associated with a particular linear acceleration component. For example, a y-axis linear acceleration orientation displacement feature can be based on a change in orientation of the y-axis acceleration component. A frequency-domain deviation feature (e.g., a user interface frequency-domain motion deviation feature, such as items vii-xii immediately above) can include a value indicative of a difference in intensity with which one or more frequencies are represented in the received motion data and a baseline.

At block 908, a notification can be generated based at least in part on the leak data from block. Generating a notification can include generating and presenting i) a visual notification (e.g., a visual presentation in a graphical user interface), ii) a haptic notification (e.g., a tactile sensation such as a vibration), iii) an audible notification (e.g., a tone being played or a synthesized voice being presented), or iv) any combination of i-iii. In some cases, a haptic notification can be provided via the control of pressurized air through the user interface. For example, to indicate to a user that a leak is occurring and can be corrected, the respiratory therapy device may provide a noticeable pulse in the air being supplied to the user interface.

In some cases, the notification can be indicative of a location of one or more unintentional leaks. For example, if location information about an unintentional leak is determined as part of block 904, the location information can be used to generate a notification in the form of a visualization of the unintentional leak on the user's smartphone or other display device.

At optional block 906, a corrective action can be determined based on the received motion data and/or the identified leak data. The corrective action can be an action determined to reduce, minimize, or eliminate the unintentional leak. In some cases, when a corrective action is determined at block 906, the notification generated at block 908 can include an indication to perform the corrected action. For example, a notification can be provided to a user to tighten or loosen a particular strap on their user interface.

Any suitable corrective action can be determined at block 906, such as i) an adjustment of the user interface; ii) an adjustment of one or more straps of the user interface; iii) a replacement of a replaceable component of the user interface with a new replaceable component; iv) a replacement of a select component of the user interface with an alternate style of the select component; v) a replacement of the user interface with an alternate type of the user interface; vi) a replacement of the user interface with an alternate size of the user interface; vii) a grooming action associated with a face of the user; viii) an adjustment of one or more parameters of a respiratory therapy device fluidly coupled to the user interface; or ix) any combination of i-viii.

In some cases, facial scan data can be acquired before or after a sleep session to optimize the selection and fit of a user interface. In some cases, such as when the corrective action includes a recommendation to replace the user interface with an alternate type or size of user interface, the facial scan data can be leveraged to select an appropriate type or size of user interface. For example, if the facial scan data originally indicates that the user would most benefit from a nasal pillow type of user interface, but the motion data collected through one or more sleep sessions shows that unintentional leaks occur, optionally despite taking other corrective actions, the system may suggest that the user switch to the next most beneficial type of user interface, such as a full face user interface.

At optional block 912, additional sensor data can be received from an additional sensor. In some cases, the additional sensor can be a remote sensor (e.g., remote from the user interface), although that need not always be the case. The additional sensor data can comprise additional motion positional and/or orientation data associated with relative movement of the user interface with respect to the motion sensor(s) acquiring the motion data from block 902. For example, an RF sensor or a LiDAR sensor can be used to identify changes in position and/or orientation of the user interface with respect to the remote sensor and/or to identify relative changes in position and/or orientation of the motion sensor(s) and the user interface with respect to the remote sensor. In other cases, the additional sensor can be associated with, or otherwise coupled to, the user interface or the motion sensor (or the component of the respiratory therapy system to which the motion sensor is coupled). The identified changes in position and/or orientation can be used to determine movement of the motion sensor(s) with respect to the user interface. The determined movement of the motion sensor(s) with respect to the user interface can be used to determine a final position and/or orientation of the motion sensor(s) with respect to the user interface. Position and/or orientation information about a motion sensor can be expressed based on a fixed frame of reference (e.g., a common frame of reference with the user interface) or a relative frame of reference (e.g., a frame of reference relative to the user interface).

In an example, a motion sensor can be coupled indirectly to a user interface via a conduit that is coupled to the user interface via a conduit cuff and/or connector. Since it may be possible for the conduit/conduit cuff, and thus the motion sensor couple thereto, to move with respect to the user interface (e.g., due to rotation of a rotating joint of the connector), additional sensor data can be acquired to determine the movement of the motion sensor with respect to the user interface (or a component thereof, e.g., the frame and/or cushion of the user interface). Thus, motion data acquired from the motion sensor can be adjusted based on this determined movement of the motion sensor with respect to the user interface to more accurately determine movement of the user interface and thus presence, and optionally intensity and/or location, of unintentional leak, such as air leak from a region of the cushion. For example, if an additional sensor determines that the motion sensor is rotating 20 degrees along a certain axis with respect to the user interface, the motion data acquired from the motion sensor can be adjusted to remove the additional 20 degrees of rotation along that certain axis.

In another example, one or more light sources coupled to the user interface can be sensed by an additional sensor (e.g., a light sensor, such as a light dependent resistor). In examples, the one or more light sources may be a surface which reflects light, such as ambient light e.g., white light or artificial light from the environment in which the sensor is located, and the surface may comprise markings varying in reflective intensity. Sensing of the light source(s) can be used to determine a position or orientation of the user interface. For example, a set of infrared light sources of known locations on the user interface can be discerned by a light sensor (e.g., an infrared sensor). The distances between the infrared light sources as sensed by the light sensor can be used to estimate an orientation of the light sources, and thus an orientation of the user interface, such as an orientation relative to the light sensor. In another example, a set of reflective markings of known locations on the user interface can be discerned by a light sensor (e.g., a light dependent resistor), and the change in position of the markings with respect to the light sensor can be used to estimate an orientation of the markings, and thus an orientation of the user interface, such as an orientation relative to the light sensor.

In some cases, the additional sensor can be coupled to the user interface, a component thereof, or a conduit/conduit cuff to read or otherwise detect a detectable element present on the conduit/conduit cuff, another component of the user interface, or the user interface. Thus, the additional sensor can be used to determine a relative movement of a motion sensor coupled to the conduit/conduit cuff with respect to the user interface or the component thereof (e.g., frame or cushion of the user interface). In an example, the additional sensor can be a light sensor used to detect light source, such as a visual element (e.g., an encoded visual element). For example, a visual pattern printed on a surface of the conduit/conduit cuff can be read by a light sensor coupled to the user interface, such that when the conduit/conduit cuff rotates with respect to the user interface, the system can determine the amount of relative rotation based on the visual pattern read by the light sensor, and thus the relative rotation of motion sensor with respect to the user interface. Other types of additional sensors and detectable elements can be used, such as a magnetic sensor (e.g., a reed switch or hall effect sensor) and a magnet.

While process 900 is depicted with certain blocks in a certain order, in some cases process 900 can be performed with additional blocks, with fewer blocks, and/or with blocks in different order. For example, in some cases, process 900 includes blocks 902, 904, 906, and 908, as well as an additional block for receiving user input indicative that the corrective action has been taken, such as to determine if the corrective action was successful in reducing, minimizing, or eliminating the unintentional leak.

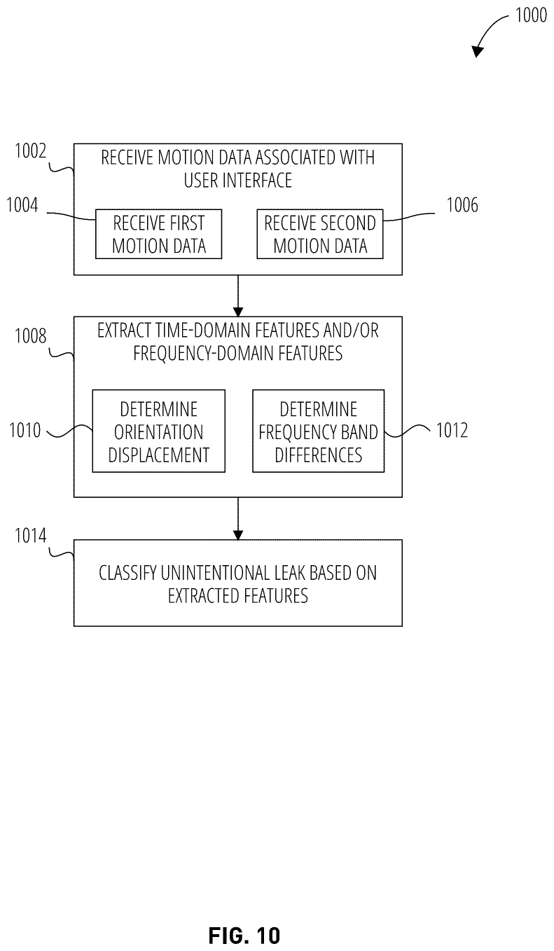

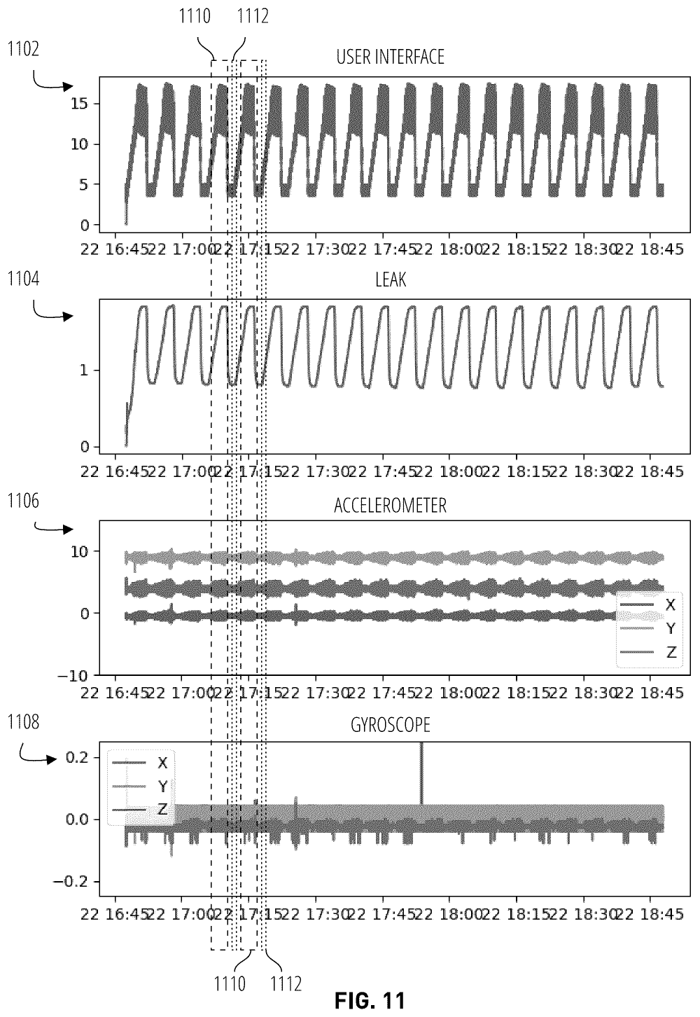

FIG. 10 is a flow chart depicting a process 1000 for analyzing user interface leak using features extracted from motion data, according to certain aspects of the present disclosure. Process 1000 can occur as part of process 900. Process 1000 can be performed using any suitable system, such as system 102 of FIG. 1.

At block 1002, motion data associated with the user interface is received. Receiving motion data at block 1002 can be similar to or the same as receiving motion data at block 902 of FIG. 9. In some cases, receiving motion data at block 1002 includes receiving first motion data at block 1004 and receiving second motion data at block 1006. First motion data can be motion data associated with little or no unintentional leakage and second motion data can be motion data associated with sufficiently high unintentional leakage or anticipated unintentional leakage.

At block 1008, time-domain features and/or frequency-domain features can be extracted from the motion data at block 1002. Extracting the time-domain features and/or frequency-domain features can include determining one or more orientation displacement features at block 1010 and/or determining one or more frequency band differences at block 1012.

Determination of an orientation displacement feature at block 1010 can include determining an average orientation of the user interface based on the first motion data from block 1004 and determining a new orientation of the user interface based on the second motion data from block 1006. The difference between the new orientation and the average orientation can be orientation displacement. This difference can be expressed according to displacement along multiple axes. In other words, the difference in orientation can be expressed as having an x-axis component, a y-axis component, and a z-axis component. One or more of the x-axis component, y-axis component, and z-axis component can be used as an orientation displacement feature.

Determination of a frequency band difference at block 1012 can include determining an average frequency signature from the first motion data from block 1004 and determining a new frequency signature from the second motion data from block 1006. The difference between these two frequency signatures can be frequency band differences. A frequency signature can be a frequency-domain representation of the motion data, indicating the intensity of different frequencies that make up the signal. In some cases, only certain frequency bands within the frequency signatures are considered. For example, in some cases only differences in frequency at approximately 14 Hz (e.g., 14 Hz, 13-15 Hz, 12-16 Hz, or 11-17 Hz) are considered for classification purposes. In some cases, multiple frequency bands are considered.

At block 1014, one or more unintentional leaks can be classified based at least in part on the extracted features from block 1010. Classifying the unintentional leak can include determining leak data indicative of the unintentional leak's classification. An unintentional leak can be classified into any suitable classes. In some cases, an unintentional leak can be classified by its location (e.g., location with respect to the user interface). In an example, an unintentional leak may be classified based on the quadrant in which the unintentional leak is located. The user interface can be considered as having four quadrants split along axes that intersect at the mouth of the user in the sagittal plane, thus defining top-left, top-right, bottom-left, and bottom-right quadrants.

For example, if a y-axis orientation displacement feature above or below a threshold value (e.g., is positive or negative), classifying the unintentional leak based on this feature may indicate that the unintentional leak is likely in one of the top or bottom quadrants, respectively. Similarly, if an x-axis orientation displacement feature is above or below a threshold value (e.g., above or below 0 or −0.5), classifying the unintentional leak additionally based on this feature may indicate that the unintentional leak is likely in a left or right quadrant. In some case, one extracted feature may depend on the results of one or more other extracted features. For example, the x-axis orientation displacement feature being above a threshold value may indicate that the unintentional leak is likely in a left quadrant if one or more other features indicate the unintentional leak is likely in a bottom quadrant, and the x-axis orientation displacement feature being above a threshold value may indicate that the unintentional leak is likely in a right quadrant if one or more other features indicate the unintentional leak is likely in a top quadrant.

Similarly, if a z-axis rotational velocity frequency band difference feature (e.g., a frequency-domain motion deviation feature) is above or below a threshold value (e.g., above or below −4.5), classifying the unintentional leak additionally based on this feature may indicate that the unintentional leak is likely in a left or right quadrant, respectively.

While process 1000 is depicted with certain blocks in a certain order, in some cases process 1000 can be performed with additional blocks, with fewer blocks, and/or with blocks in different order. For example, in some cases, block 1008 includes block 1012 but not block 1010. As another example, in some cases, motion data received at block 1002 can include receiving second motion data at block 1006 without receiving first motion data at block 1004.

FIG. 11 is a set of charts depicting collected data from a user interface exhibiting unintentional leakage, according to certain aspects of the present disclosure. The set of charts depicted in FIG. 11 is associated with a first set of experimental data according to certain aspects of the present disclosure with an unintentional leak implemented due to an overly loose bottom-left strap of the user interface. The set of charts can include a user interface pressure chart 1102, a leak chart 1104, an accelerometer chart 1106, and a gyroscope chart 1108.

The user interface pressure chart 1102 shows the pressure of the air supplied to the user interface in cm H2O. The repeated rising and falling of the pressure seen on the user interface pressure chart 1102 shows how the supplied air was repeatedly driven at low and high pressures to cause an intensity of the unintentional leak to decrease and increase, respectively, as depicted in the leak chart 1104. The line in the leak chart 1104 indicates the intensity of the unintentional leak. The boxes marked by 1110 and 1112 indicate regions of high leakage and low leakage, respectively.

The accelerometer chart 1106 and gyroscope chart 1108 depict motion data acquired during these times. As seen in the accelerometer chart 1106 and gyroscope chart 1108, certain patterns appear in acceleration and rotational velocity, respectively, during periods of high leak 1110 and periods of low leak 1112. These patterns can be further analyzed to facilitate classifying unintentional leaks as being associated with a bottom-left strap being loose. In some cases, the periods of low leak 1112 can be used as a baseline to which the periods of high leak 1110 can be compared.

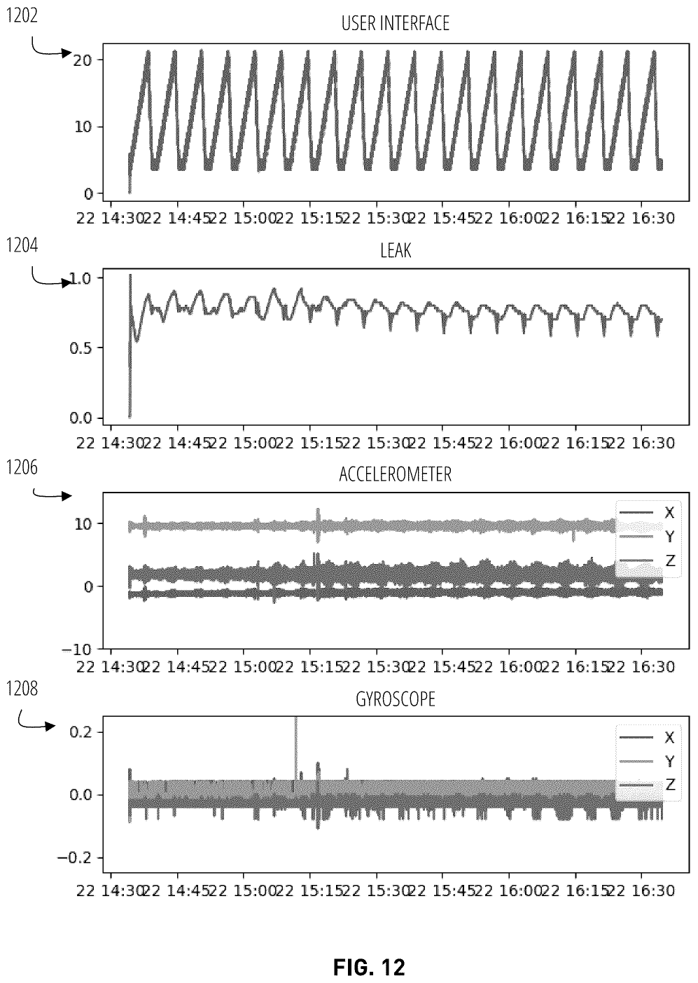

FIG. 12 is a set of charts depicting collected data from a user interface exhibiting unintentional leaks, according to certain aspects of the present disclosure. The set of charts depicted in FIG. 12 is associated with a second set of experimental data according to certain aspects of the present disclosure with an unintentional leak implemented due to an overly loose top-right strap of the user interface. The set of charts can include a user interface user interface pressure chart 1202, a leak chart 1204, an accelerometer chart 1206, and a gyroscope chart 1208.

The user interface pressure chart 1202 shows the pressure of the air supplied to the user interface in cm H2O. The repeated rising and falling of the pressure seen on the user interface user interface pressure chart 1202 shows how the supplied air was repeatedly driven at low and high pressures to cause an intensity of the unintentional leak to decrease and increase, respectively, as depicted in the leak chart 1204. The line in the leak chart 1204 indicates the intensity of the unintentional leak. The boxes marked by 1210 and 1212 indicate regions of high leakage and low leakage, respectively.

The accelerometer chart 1206 and gyroscope chart 1208 depict motion data acquired during these times. As seen in the accelerometer chart 1206 and gyroscope chart 1208, certain patterns appear in acceleration and rotational velocity, respectively, during periods of high leak 1210 and periods of low leak 1212. These patterns can be further analyzed to facilitate classifying unintentional leaks as being associated with a bottom-left strap being loose. In some cases, the periods of low leak 1212 can be used as a baseline to which the periods of high leak 1210 can be compared.

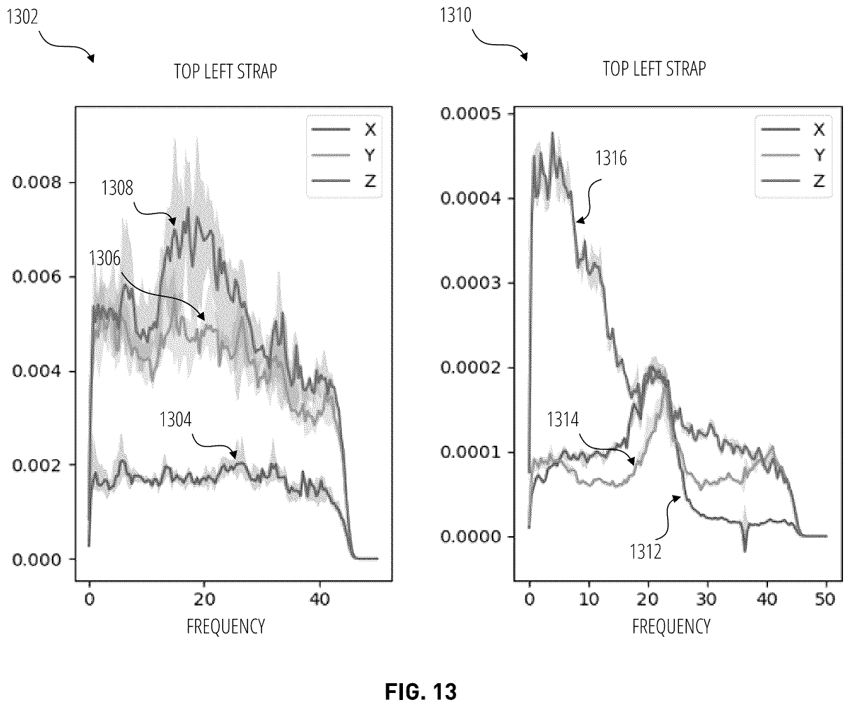

FIG. 13 is a set of charts 1302, 1310 depicting frequency-domain acceleration data associated with unintentional leaks at different locations with respect to a user interface, according to certain aspects of the present disclosure. Chart 1302 depicted in FIG. 13 is associated with a third set of experimental data according to certain aspects of the present disclosure with an unintentional leak implemented due to an overly loose top-left strap of the user interface. Chart 1310 depicted in FIG. 13 is associated with a fourth set of experimental data according to certain aspects of the present disclosure with an unintentional leak implemented due to an overly loose bottom-right strap of the user interface.

Chart 1302 shows, for a loose top-left strap, the frequency-domain (e.g., via fast Fourier transform) data for different axes of acceleration as detected by the motion sensor. For example, line 1304 shows the frequency spectrum for x-axis acceleration, line 1306 shows the frequency spectrum for y-axis acceleration, and line 1308 shows the frequency spectrum for z-axis acceleration.

Chart 1310 shows, for a loose bottom-right strap, the frequency-domain (e.g., via fast Fourier transform) data for different axes of acceleration as detected by the motion sensor. For example, line 1312 shows the frequency spectrum for x-axis acceleration, line 1314 shows the frequency spectrum for y-axis acceleration, and line 1316 shows the frequency spectrum for z-axis acceleration.

It can be seen from charts 1302, 1310 that different straps being loose exhibit different frequency-domain signatures in acceleration data.

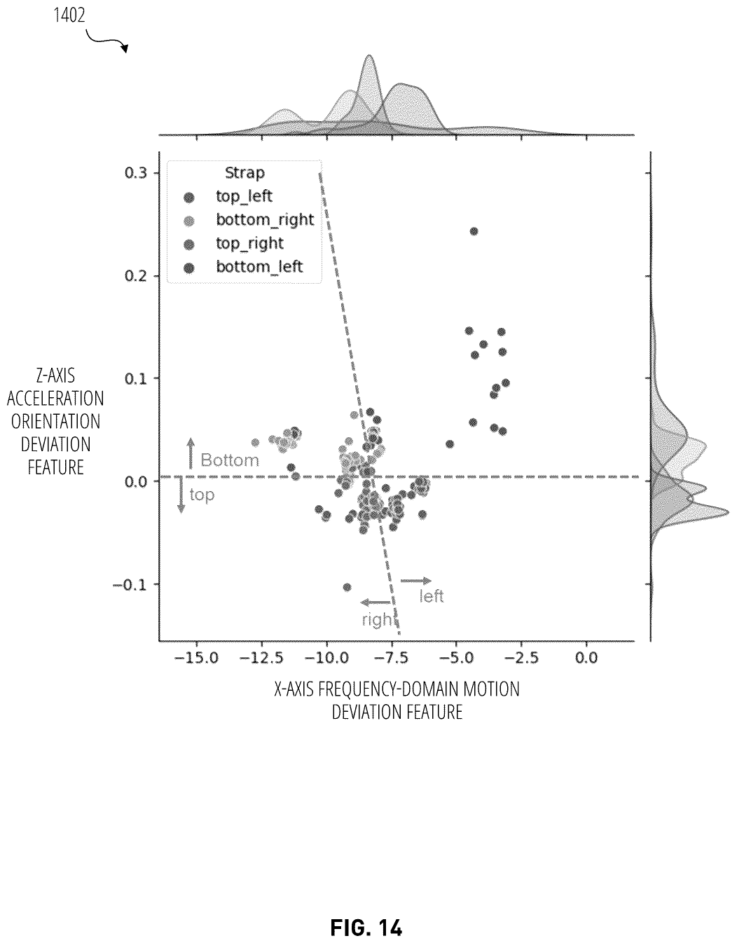

FIG. 14 is a chart 1402 depicting classification of unintentional leaks based on two features, using a user-interface-mounted sensor, according to certain aspects of the present disclosure. Specifically, the chart 1402 depicts a z-axis acceleration orientation displacement feature and an x-axis frequency-domain motion deviation feature being used to classify a series of experimental trials with different straps being loosened to cause unintentional leak.

As seen in chart 1402, the z-axis acceleration orientation displacement feature can reliably differentiate between top quadrants and bottom quadrants, while the z-axis frequency-domain motion deviation feature shows strong performance in differentiating between left and right quadrants. As depicted in chart 1402, the threshold value for differentiating between left and right for the z-axis frequency-domain motion deviation feature varies depending on the value of the z-axis acceleration orientation displacement feature.

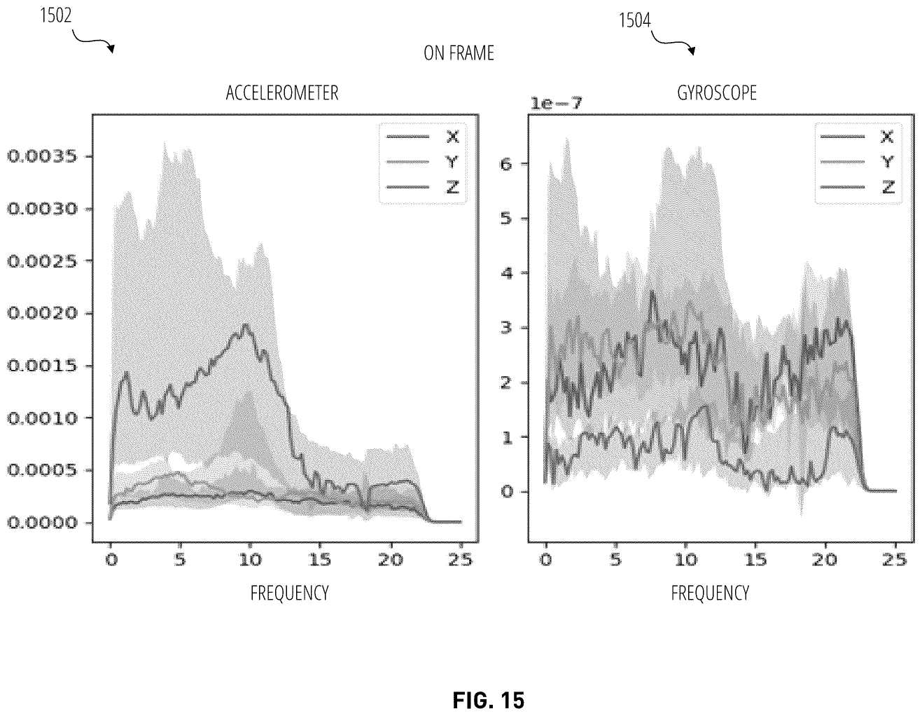

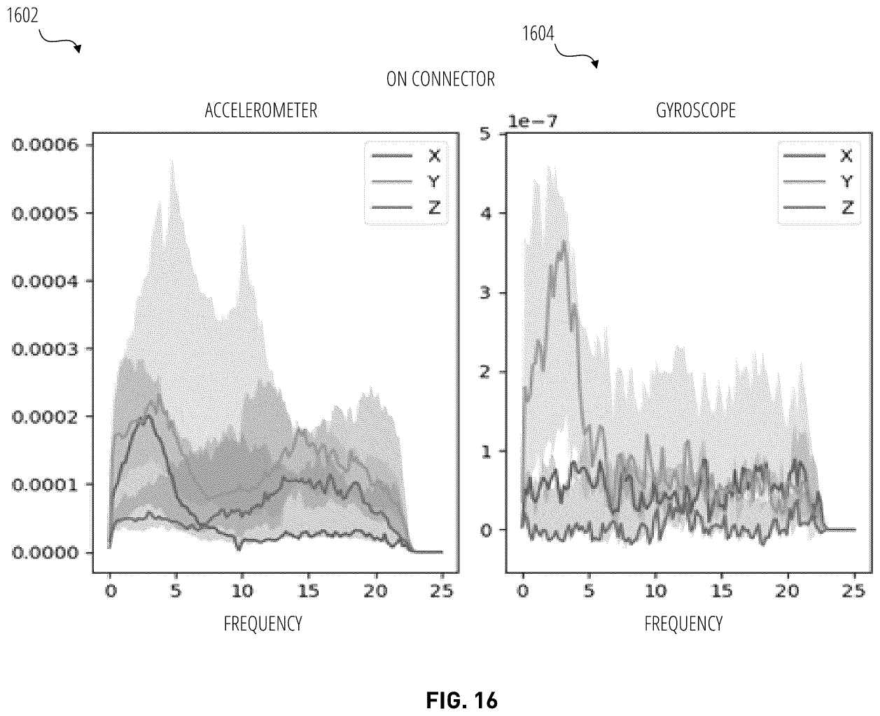

FIG. 15 is a set of charts 1502, 1504 depicting frequency-domain acceleration data and frequency-domain rotational velocity data associated with an unintentional leak as detected from an accelerometer and gyroscope located on a frame of a user interface, according to certain aspects of the present disclosure. Chart 1502 shows certain patterns (e.g., signatures) in the frequency-domain acceleration data. Chart 1504 shows certain patterns in the frequency-domain rotational velocity data. These various patterns can be used in the differentiation and classification of unintentional leaks as disclosed in further detail herein.

FIG. 16 is a set of charts 1602, 1604 depicting frequency-domain acceleration data and frequency-domain rotational velocity data associated with an unintentional leak as detected from an accelerometer and gyroscope located on an elbow connector of a user interface, according to certain aspects of the present disclosure. The underlying motion data used for charts 1602, 1604 is the same as that used for charts 1502, 1504 of FIG. 15.

Chart 1602 shows certain patterns in the frequency-domain acceleration data. Chart 1604 shows certain patterns in the frequency-domain rotational velocity data. These various patterns can be used in the differentiation and classification of unintentional leaks as disclosed in further detail herein.

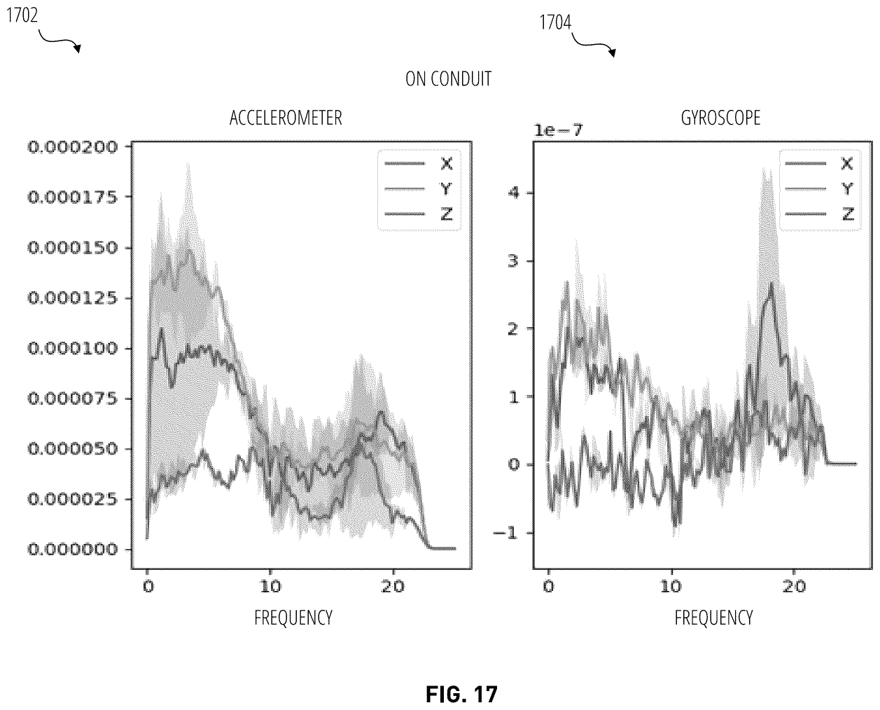

FIG. 17 is a set of charts 1702, 1704 depicting frequency-domain acceleration data and frequency-domain rotational velocity data associated with an unintentional leak as detected from an accelerometer and gyroscope located on a conduit cuff coupled to a user interface, according to certain aspects of the present disclosure. The underlying motion data used for charts 1702, 1704 is the same as that used for charts 1502, 1504 of FIG. 15.

Chart 1702 shows certain patterns in the frequency-domain acceleration data. Chart 1704 shows certain patterns in the frequency-domain rotational velocity data. These various patterns can be used in the differentiation and classification of unintentional leaks as disclosed in further detail herein.

It can be seen that frequency-domain motion data signatures remain present between charts 1502, 1602, 1702 and between charts 1504, 1604, 1704, despite the motion data originating from a motion sensor on the frame of the user interface, on the connector of the user interface, and on a conduit coupled to the user interface, respectively. Thus, while better results may be achieved by a motion sensor coupled directly to the user interface, sufficiently good results can be achieved by a motion sensor coupled indirectly to the user interface.

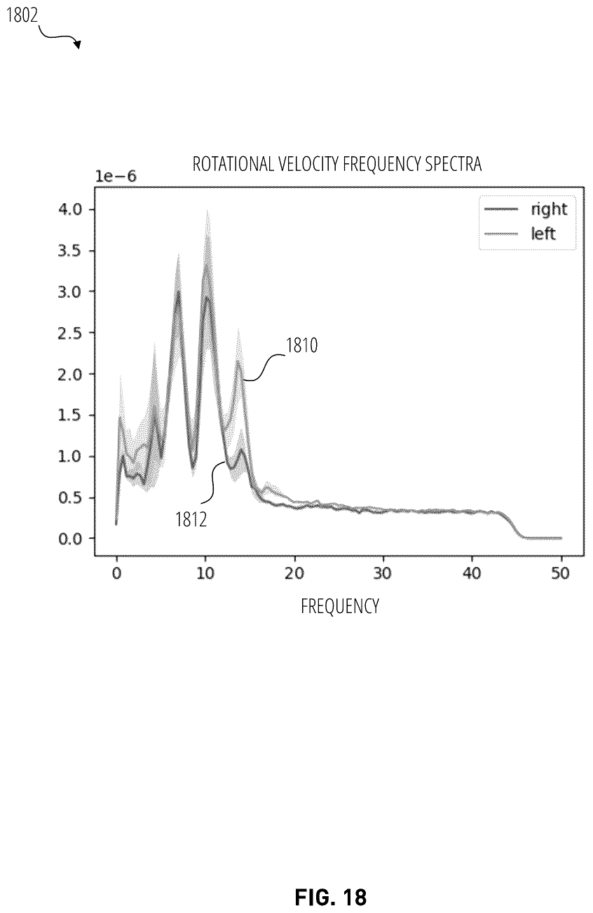

FIG. 18 is a chart 1802 depicting frequency-domain rotational velocity data used to differentiate straps of a user interface likely to be causing an unintentional leak, according to certain aspects of the present disclosure. Chart 1802 is prepared from a set of experimental trials. Line 1810 shows the average frequency-domain spectral intensity of the z-axis rotational velocity for unintentional leaks caused by a left strap being loose. Line 1812 shows the average frequency-domain spectral intensity of the z-axis rotational velocity for unintentional leaks caused by a right strap being loose.

As seen in chart 1802, lines 1810, 1812 follow closely with one another at some frequencies, but vary significantly at other frequencies. Thus, the frequency-domain spectral intensity for z-axis rotational velocity at certain frequencies (e.g., at or around 14 Hz) can be useful for differentiating between left straps and right straps.

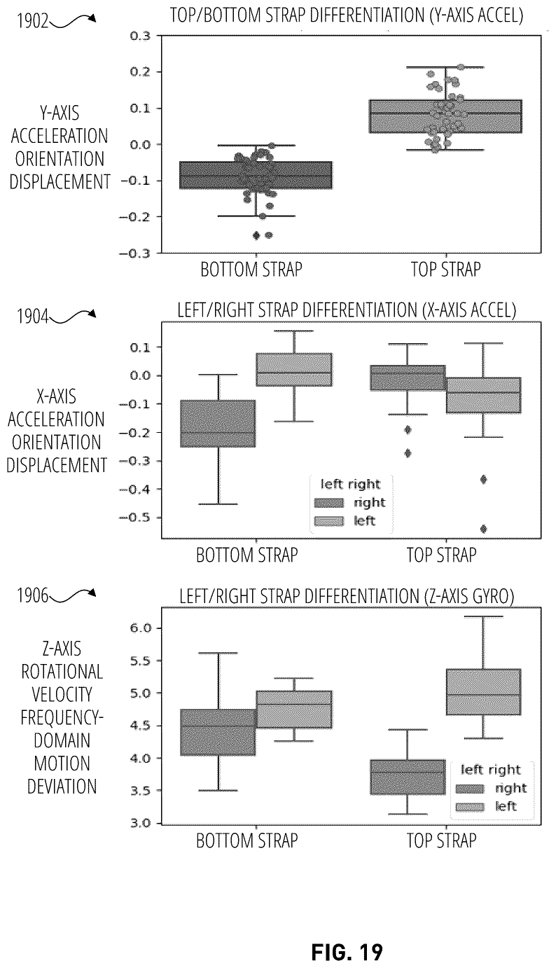

FIG. 19 is a set of charts depicting various features of motion data used to differentiate straps of a user interface likely to be causing an unintentional leak, according to certain aspects of the present disclosure. Charts 1902, 1904, 1906 are prepared from the same set of experimental trials as chart 1802 of FIG. 18.

Chart 1902 shows how a y-axis acceleration orientation displacement feature can be used to separate between bottom and top straps.

Chart 1904 shows how a y-axis acceleration orientation displacement feature can be used to separate between left and right straps. In some cases, the manner in which the y-axis acceleration orientation displacement feature separates between left and right changes depending on one or more other features having been used to determine that the strap in question is a bottom strap or a top strap.

Chart 1906 shows how a z-axis rotational velocity frequency-domain motion deviation feature can be used to separate between left and right straps. In some cases, the manner in which the z-axis rotational velocity frequency-domain motion deviation feature separates between left and right changes depending on one or more other features having been used to determine that the strap in question is a bottom strap or a top strap.

As seen in chart 1904, the y-axis acceleration orientation displacement feature is better at separating bottom-left and bottom-right straps than top-left and top-right straps. Likewise, chart 1906 shows that the z-axis rotational velocity frequency-domain motion deviation feature is better at separating top-left and top-right straps than bottom-left and bottom-right straps. Thus, when one or more features (e.g., a y-axis acceleration orientation displacement feature) indicate that the strap in question is likely a bottom strap, the y-axis acceleration orientation displacement feature may be used to determine if it is a bottom-left strap or a bottom-right strap, whereas when the one or more features indicate that the strap in question is likely a top strap, the z-axis rotational velocity frequency-domain motion deviation feature may be used to determine if it is a top-left strap or a top-right strap.

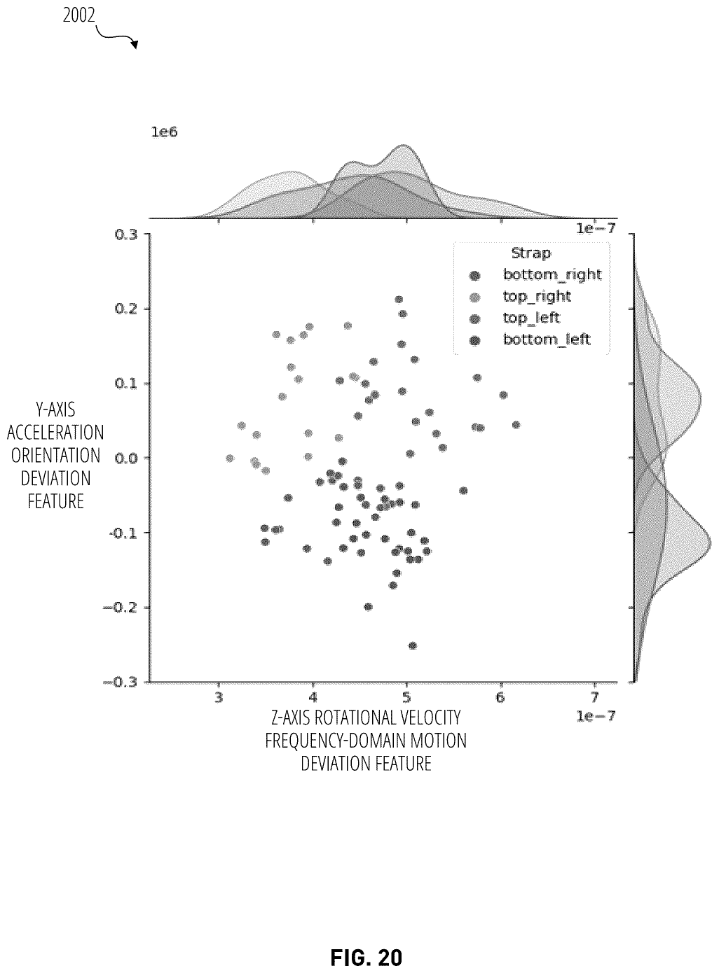

FIG. 20 is a chart 2002 depicting classification of unintentional leaks based on two features using a conduit-cuff-mounted sensor, according to certain aspects of the present disclosure. Specifically, the chart 2002 depicts a y-axis acceleration orientation displacement feature and a z-axis rotational velocity frequency-domain motion deviation feature being used to classify a series of experimental trials with different straps being loosened to cause unintentional leak.

As seen in chart 2002, the y-axis acceleration orientation displacement feature can reliably differentiate between top quadrants and bottom quadrants, while the z-axis rotational velocity frequency-domain motion deviation feature shows strong performance in differentiating between left and right quadrants, especially with respect to the top-left and top-right quadrants.

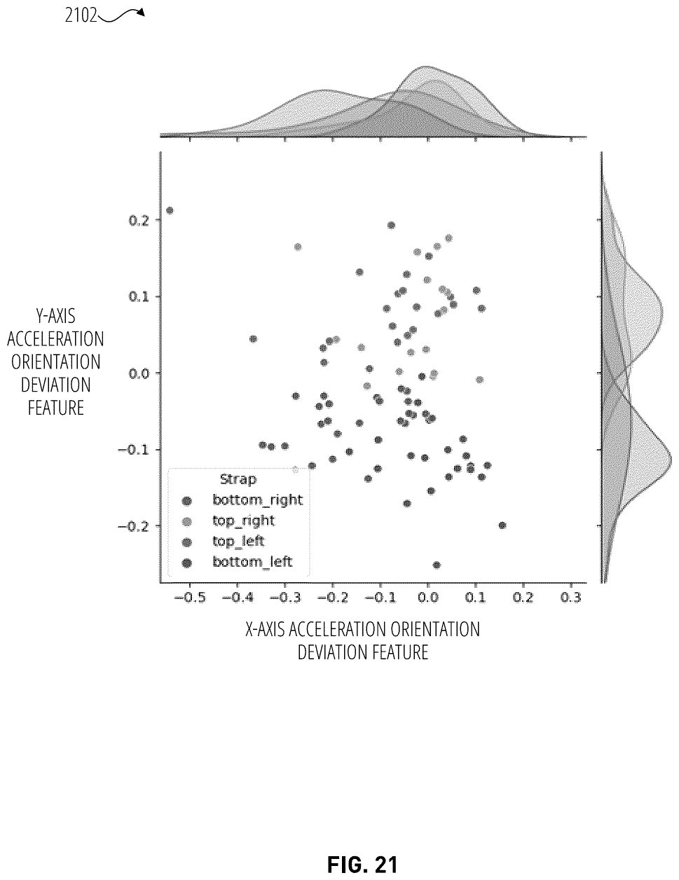

FIG. 21 is a chart 2102 depicting classification of unintentional leaks based on two features, according to certain aspects of the present disclosure. Specifically, the chart 2102 depicts a y-axis acceleration orientation displacement feature and an x-axis acceleration orientation displacement feature being used to classify a series of experimental trials with different straps being loosened to cause unintentional leak.

As seen in chart 2102, the y-axis acceleration orientation displacement feature can reliably differentiate between top quadrants and bottom quadrants, while the x-axis acceleration orientation displacement feature shows strong performance in differentiating between left and right quadrants, especially with respect to the bottom-left and bottom-right quadrants.

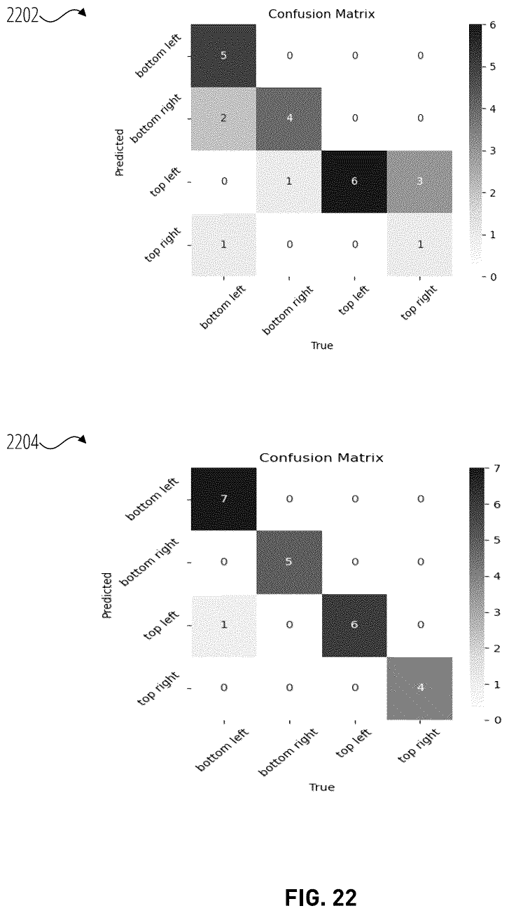

FIG. 22 is a set of confusion matrix charts 2202, 2204 depicting classification accuracy when different numbers of features are used to classify the unintentional leak, according to certain aspects of the present disclosure. The classifications presented in confusion matrix charts 2202, 2204 are shown as bottom left, bottom right, top left, and top right, representing the different quadrants in which the unintentional leak is occurring (“true” values along the x-axis) or predicted to be occurring (e.g., “predicted” values along the y-axis), such as via process 900 of FIG. 9. The confusion matrix charts 2202, 2204 were generated using a set of experimental data according to certain aspects of the present disclosure.

Any number of features from motion data can be used to classify an unintentional leak. Examples of such features include i) an x-axis linear acceleration orientation displacement feature; ii) a y-axis linear acceleration orientation displacement feature; iii) a z-axis linear acceleration orientation displacement feature; iv) an x-axis rotational velocity displacement feature; v) a y-axis rotational velocity displacement feature; vi) a z-axis rotational velocity displacement feature; vii) an x-axis linear acceleration frequency-domain deviation feature; viii) a y-axis linear acceleration frequency-domain deviation feature; ix) a z-axis linear acceleration frequency-domain deviation feature; x) an x-axis rotational velocity frequency-domain deviation feature; xi) a y-axis rotational velocity frequency-domain deviation feature; xii) a z-axis rotational velocity frequency-domain deviation feature; or xiii) any combination of i-xii.

The three-feature confusion matrix chart 2202 shows the accuracy achieved when classification is performed using i) a y-axis orientation displacement feature, ii) an x-axis orientation displacement feature, and iii) a rotational velocity frequency-domain deviation feature. In such cases, the y-axis orientation displacement feature can be especially useful at indicating whether the unintentional leak is near the top or bottom of the user interface, the x-axis orientation displacement feature can be especially useful at indicating whether the unintentional leak is near the left or right side of the user interface, and the rotational velocity frequency-domain motion deviation feature can be especially useful at indicating whether the unintentional leak is near the left or right side of the user interface.

The all-feature confusion matrix chart 2204 shows the accuracy achieved when classification is performed using all of the aforementioned orientation displacement features and frequency-domain deviation features, including: i) an x-axis linear acceleration orientation displacement feature; ii) a y-axis linear acceleration orientation displacement feature; iii) a z-axis linear acceleration orientation displacement feature; iv) an x-axis rotational velocity displacement feature; v) a y-axis rotational velocity displacement feature; vi) a z-axis rotational velocity displacement feature; vii) an x-axis linear acceleration frequency-domain deviation feature; viii) a y-axis linear acceleration frequency-domain deviation feature; ix) a z-axis linear acceleration frequency-domain deviation feature; x) an x-axis rotational velocity frequency-domain deviation feature; xi) a y-axis rotational velocity frequency-domain deviation feature; and xii) a z-axis rotational velocity frequency-domain deviation feature.

As seen in the confusion matrix charts 2202, 2204, the use of the three identified features in classifying unintentional leaks can achieve good accuracy of approximately 70%, and the use of all identified features can further improve the accuracy up to 96%.

As used below, any reference to a series of implementations is to be understood as a reference to each of those implementations disjunctively (e.g., “Implementations 1-4” is to be understood as “Implementation 1, 2, 3, or 4”).

Implementation 1 is a method for analyzing user interface leakage, comprising: receiving, at a computing device, motion data associated with orientation of a user interface worn by a user during a sleep session; analyzing the motion data to identify leak data, the leak data indicative of at least one unintentional leak from the user interface; and generating a notification based at least in part on the leak data, the notification indicative of the presence of the at least one unintentional leak.

Implementation 2 is the method of implementation(s) 1, wherein receiving the motion data includes receiving the motion data from one or more accelerometers coupled to the user interface.

Implementation 3 is the method of implementation(s) 2, wherein the one or more accelerometers includes i) an accelerometer directly coupled to the user interface; ii) an accelerometer coupled to a connector coupled to the user interface; iii) an accelerometer coupled to a conduit coupled to the user interface; or iv) any combination of i-iii.

Implementation 4A is the method of implementation(s) 2 or 3, further comprising: receiving additional sensor data associated with an orientation of the user interface during the sleep session; and determining movement of the one or more accelerometers with respect to the user interface based at least in part on the additional sensor data.

Implementation 4B is the method of implementation(s) 2 or 3, further comprising: receiving additional sensor data indicative of relative movement of the user interface with respect to the one or more accelerometers during the sleep session, wherein analyzing the motion data to identify the leak data is based at least in part on the additional sensor data.

Implementation 5 is the method of implementation(s) 4A or 4B, wherein the additional sensor data is indicative of an orientation of a light source coupled to the user interface.

Implementation 6 is the method of implementation(s) 4A or 4B, wherein the additional sensor data is light sensor data of an encoded visual element associated with the user interface, wherein the light sensor data is indicative of the orientation of the user interface.

Implementation 7 is the method of any one of implementation(s)s 1 to 6, wherein the notification is further indicative of a location of the at least one unintentional leak with reference to the user interface.

Implementation 8 is the method of any one of implementation(s)s 1 to 7, further comprising determining, based at least in part on the leak data, a corrective action for reducing the at least one unintentional leak, wherein the notification includes an indication to perform the corrective action.

Implementation 9 is the method of implementation(s) 8, wherein the corrective action includes i) an adjustment of the user interface; ii) an adjustment of one or more straps of the user interface; iii) a replacement of a replaceable component of the user interface with a new replaceable component; iv) a replacement of a select component of the user interface with an alternate style of the select component; v) a replacement of the user interface with an alternate type of the user interface; vi) a replacement of the user interface with an alternate size of the user interface; vii) a grooming action associated with a face of the user; viii) an adjustment of one or more parameters of a respiratory therapy device fluidly coupled to the user interface; or ix) any combination of i-viii.

Implementation 10 is the method of any one of implementation(s)s 1 to 9, wherein analyzing the motion data to identify leak data includes identifying body position data indicative of a body position of the user based at least in part on the motion data.

Implementation 11 is the method of any one of implementation(s)s 1 to 10, wherein analyzing the motion data to identify leak data includes: extracting a first portion of the motion data assumed to be associated with low leakage or no leakage; generating a baseline signal based at least in part on the first portion of the motion data; and identifying the at least one unintentional leak when the motion data deviates from the baseline signal by at least a threshold value.

Implementation 12 is the method of any one of implementation(s)s 1 to 11, wherein analyzing the motion data to identify leak data includes identifying the at least one unintentional leak when the motion data deviates from a baseline signal by at least a threshold value, wherein the baseline signal is based at least in part on a portion of historical motion data assumed to be associated with low leakage or no leakage, the historical motion data associated with a prior sleep session.

Implementation 13 is the method of any one of implementation(s)s 1 to 12, wherein analyzing the motion data includes: extracting frequency-domain motion data from the motion data; and identifying the at least one unintentional leak based at least in part on the frequency-domain motion data.

Implementation 14 is the method of implementation(s) 13, wherein the frequency-domain motion data includes at least a first portion of the frequency-domain motion data associated with acceleration in a first direction and at least a second portion of the frequency-domain motion data associated with acceleration in a second direction that is orthogonal to the first direction.

Implementation 15 is the method of implementation(s) 14, wherein identifying the at least one unintentional leak based at least in part on the frequency-domain motion data includes determining location information for each of the at least one unintentional leak based at least in part on the first portion of the frequency-domain motion data and the second portion of the frequency-domain motion data.

Implementation 16 is the method of implementation(s) 15, wherein generating the notification includes generating an indication of a location of each of the at least one unintentional leak based at least in part on the determined location information.

Implementation 17 is the method of implementation(s) 15 or 16, further comprising determining a corrective action for reducing the at least one unintentional leak based at least in part on the determined location information, wherein the notification includes an indication to perform the corrective action.

Implementation 18 is the method of any one of implementation(s)s 13 to 17, wherein the frequency-domain motion data includes frequency-domain linear acceleration data, and wherein identifying the at least one unintentional leak is based at least in part on the frequency-domain linear acceleration data.

Implementation 19 is the method of any one of implementation(s)s 13 to 18, wherein the frequency-domain motion data includes frequency-domain rotational velocity data, and wherein identifying the at least one unintentional leak is based at least in part on the frequency-domain rotational velocity data.

Implementation 20 is the method of any one of implementation(s)s 1 to 19, wherein analyzing the motion data includes: determining average motion data from the motion data, the average motion data indicative of an average orientation of the user interface with respect to the face of the user; identifying a deviation in orientation of the user interface from the average orientation based at least in part on the motion data, the deviation being greater than a threshold value; and identifying the at least one unintentional leak based at least in part on the identified deviation.

Implementation 21 is the method of any one of implementation(s)s 1 to 20, wherein analyzing the motion data to identify leak data includes: extracting a plurality of motion data features from the motion data, including at least i) a user interface orientation displacement feature, and ii) a frequency-domain motion deviation feature; identifying the at least one unintentional leak based at least in part on the user interface orientation displacement feature and the frequency-domain motion deviation feature.

Implementation 22 is the method of any one of implementation(s)s 1 to 21, wherein analyzing the motion data includes: extracting frequency-domain motion data from the motion data; and identifying the at least one unintentional leak based at least in part on the frequency-domain motion data.

Implementation 23 is the method of any one of implementation(s)s 1 to 22, wherein the motion data includes linear acceleration data.

Implementation 24 is the method of any one of implementation(s)s 1 to 23, wherein the motion data includes angular velocity data.

Implementation 25 is a system comprising: a control a control system comprising one or more processors; and a memory having stored thereon machine readable instructions; wherein the control system is coupled to the memory, and the method of any one of implementation(s)s 1 to 24 is implemented when the machine executable instructions in the memory are executed by at least one of the one or more processors of the control system.

Implementation 26 is a system for analyzing user interface leakage, the system comprising a control system configured to implement the method of any one of implementation(s)s 1 to 24.

Implementation 27 is a computer program product comprising instructions which, when executed by a computer, cause the computer to carry out the method of any one of implementation(s)s 1 to 24.

Implementation 28 is the computer program product of implementation(s) 27, wherein the computer program product is a non-transitory computer readable medium.

Implementation 29 is a system comprising: one or more motion sensors coupled to a user interface worn by a user during a sleep session, the user interface fluidly coupled to a respiratory therapy device for providing a flow of air from the respiratory therapy device to a respiratory system of the user; one or more processors; and a non-transitory computer-readable storage medium containing instructions which, when executed on the one or more processors, cause the one or more processors to perform operations including: receiving, at a computing device, motion data associated with orientation of the user interface; analyzing the motion data to identify leak data, the leak data indicative of at least one unintentional leak from the user interface; and generating a notification based at least in part on the leak data, the notification indicative of the presence of the at least one unintentional leak.

Implementation 30 is the system of implementation(s) 29, wherein the one or more motion sensors includes one or more accelerometers, and wherein receiving the motion data includes receiving the motion data from the one or more accelerometers.

Implementation 31 is the system of implementation(s) 30, wherein the one or more accelerometers includes i) an accelerometer directly coupled to the user interface; ii) an accelerometer coupled to a connector coupled to the user interface; iii) an accelerometer coupled to a conduit coupled to the user interface; or iv) any combination of i-iii.

Implementation 32A is the system of implementation(s) 30 or 31, wherein the operations further include: receiving additional sensor data associated with an orientation of the user interface during the sleep session; and determining movement of the one or more accelerometers with respect to the user interface based at least in part on the additional sensor data.

Implementation 32B is the method of implementation(s) 30 or 31, further comprising: receiving additional sensor data indicative of relative movement of the user interface with respect to the one or more accelerometers during the sleep session, wherein analyzing the motion data to identify the leak data is based at least in part on the additional sensor data.

Implementation 33 is the system of implementation(s) 32A or 32B, wherein the additional sensor data is indicative of an orientation of a light source coupled to the user interface.

Implementation 34 is the system of implementation(s) 32A or 32B, wherein the additional sensor data is light sensor data of an encoded visual element associated with the user interface, wherein the light sensor data is indicative of the orientation of the user interface.

Implementation 35 is the system of implementation(s) 29 to 34, wherein the notification is further indicative of a location of the at least one unintentional leak with reference to the user interface.

Implementation 36 is the system of implementation(s) 29 to 35, wherein the operations further include determining, based at least in part on the leak data, a corrective action for reducing the at least one unintentional leak, wherein the notification includes an indication to perform the corrective action.

Implementation 37 is the system of implementation(s) 36, wherein the corrective action includes i) an adjustment of the user interface; ii) an adjustment of one or more straps of the user interface; iii) a replacement of a replaceable component of the user interface with a new replaceable component; iv) a replacement of a select component of the user interface with an alternate style of the select component; v) a replacement of the user interface with an alternate type of the user interface; vi) a replacement of the user interface with an alternate size of the user interface; vii) a grooming action associated with a face of the user; viii) an adjustment of one or more parameters of a respiratory therapy device fluidly coupled to the user interface; or ix) any combination of i-viii.

Implementation 38 is the system of implementation(s) 29 to 37, wherein analyzing the motion data to identify leak data includes identifying body position data indicative of a body position of the user based at least in part on the motion data.

Implementation 39 is the system of implementation(s) 29 to 38, wherein analyzing the motion data to identify leak data includes: extracting a first portion of the motion data assumed to be associated with low leakage or no leakage; generating a baseline signal based at least in part on the first portion of the motion data; and identifying the at least one unintentional leak when the motion data deviates from the baseline signal by at least a threshold value.

Implementation 40 is the system of implementation(s) 29 to 39, wherein analyzing the motion data to identify leak data includes identifying the at least one unintentional leak when the motion data deviates from a baseline signal by at least a threshold value, wherein the baseline signal is based at least in part on a portion of historical motion data assumed to be associated with low leakage or no leakage, the historical motion data associated with a prior sleep session.

Implementation 41 is the system of implementation(s) 29 to 40, wherein analyzing the motion data includes: extracting frequency-domain motion data from the motion data; and identifying the at least one unintentional leak based at least in part on the frequency-domain motion data.

Implementation 42 is the system of implementation(s) 41, wherein the frequency-domain motion data includes at least a first portion of the frequency-domain motion data associated with acceleration in a first direction and at least a second portion of the frequency-domain motion data associated with acceleration in a second direction that is orthogonal to the first direction.

Implementation 43 is the system of implementation(s) 42, wherein identifying the at least one unintentional leak based at least in part on the frequency-domain motion data includes determining location information for each of the at least one unintentional leak based at least in part on the first portion of the frequency-domain motion data and the second portion of the frequency-domain motion data.

Implementation 44 is the system of implementation(s) 43, wherein generating the notification includes generating an indication of a location of each of the at least one unintentional leak based at least in part on the determined location information.

Implementation 45 is the system of implementation(s) 43 or 44, wherein the operations further include determining a corrective action for reducing the at least one unintentional leak based at least in part on the determined location information, wherein the notification includes an indication to perform the corrective action.

Implementation 46 is the system of implementation(s) 41 to 45, wherein the frequency-domain motion data includes frequency-domain linear acceleration data, and wherein identifying the at least one unintentional leak is based at least in part on the frequency-domain linear acceleration data.

Implementation 47 is the system of implementation(s) 41 to 46, wherein the frequency-domain motion data includes frequency-domain rotational velocity data, and wherein identifying the at least one unintentional leak is based at least in part on the frequency-domain rotational velocity data.

Implementation 48 is the system of implementation(s) 29 to 47, wherein analyzing the motion data includes: determining average motion data from the motion data, the average motion data indicative of an average orientation of the user interface with respect to the face of the user; identifying a deviation in orientation of the user interface from the average orientation based at least in part on the motion data, the deviation being greater than a threshold value; and identifying the at least one unintentional leak based at least in part on the identified deviation.

Implementation 49 is the system of implementation(s) 29 to 48, wherein analyzing the motion data to identify leak data includes: extracting a plurality of motion data features from the motion data, including at least i) a user interface orientation displacement feature, and ii) a frequency-domain motion deviation feature; identifying the at least one unintentional leak based at least in part on the user interface orientation displacement feature and the frequency-domain motion deviation feature.

Implementation 50 is the system of implementation(s) 29 to 49, wherein analyzing the motion data includes: extracting frequency-domain motion data from the motion data; and identifying the at least one unintentional leak based at least in part on the frequency-domain motion data.

Implementation 51 is the system of implementation(s) 29 to 50, wherein the motion data includes linear acceleration data.

Implementation 52 is the system of implementation(s) 29 to 51, wherein the motion data includes angular velocity data.

One or more elements or aspects or steps, or any portion(s) thereof, from one or more of any of the above implementations can be combined with one or more elements or aspects or steps, or any portion(s) thereof, from one or more of any of the other implementations or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method for analyzing user interface leakage, comprising:
receiving, at a computing device, motion data associated with orientation of a user interface worn by a user during a sleep session;
analyzing the motion data to identify leak data, the leak data indicative of at least one unintentional leak from the user interface; and
generating a notification based at least in part on the leak data, the notification indicative of the presence of the at least one unintentional leak.

2. The method of claim 1, wherein receiving the motion data includes receiving the motion data from one or more accelerometers coupled to the user interface.

3. The method of claim 2, wherein the one or more accelerometers includes i) an accelerometer directly coupled to the user interface; ii) an accelerometer coupled to a connector coupled to the user interface; iii) an accelerometer coupled to a conduit coupled to the user interface; or iv) any combination of i-iii.

4. The method of claim 2, further comprising receiving additional sensor data indicative of relative movement of the user interface with respect to the one or more accelerometers during the sleep session, wherein analyzing the motion data to identify the leak data is based at least in part on the additional sensor data.

5. The method of claim 4, wherein the additional sensor data is light sensor data of an encoded visual element associated with the user interface, wherein the light sensor data is indicative of the orientation of the user interface.

6. The method of claim 1, further comprising determining, based at least in part on the leak data, a corrective action for reducing the at least one unintentional leak, wherein the notification includes an indication to perform the corrective action.

7. The method of claim 6, wherein the corrective action includes i) an adjustment of the user interface; ii) an adjustment of one or more straps of the user interface; iii) a replacement of a replaceable component of the user interface with a new replaceable component; iv) a replacement of a select component of the user interface with an alternate style of the select component; v) a replacement of the user interface with an alternate type of the user interface; vi) a replacement of the user interface with an alternate size of the user interface; vii) a grooming action associated with a face of the user; viii) an adjustment of one or more parameters of a respiratory therapy device fluidly coupled to the user interface; or ix) any combination of i-viii.

8. The method of claim 1, wherein analyzing the motion data to identify the leak data includes:
extracting a first portion of the motion data assumed to be associated with low leakage or no leakage;
generating a baseline signal based at least in part on the first portion of the motion data; and
identifying the at least one unintentional leak when the motion data deviates from the baseline signal by at least a threshold value.

9. The method of claim 1, wherein analyzing the motion data to identify the leak data includes identifying the at least one unintentional leak when the motion data deviates from a baseline signal by at least a threshold value, wherein the baseline signal is based at least in part on a portion of historical motion data assumed to be associated with low leakage or no leakage, the historical motion data associated with a prior sleep session.

10. The method of claim 1, wherein analyzing the motion data includes:
extracting frequency-domain motion data from the motion data, the frequency-domain motion data including (i) frequency-domain linear acceleration data; (ii) frequency-domain rotational acceleration data; (iii) or (i) and (ii); and
identifying the at least one unintentional leak based at least in part on the frequency-domain motion data.

11. The method of claim 10, wherein the frequency-domain motion data includes at least a first portion of the frequency-domain motion data associated with acceleration in a first direction and at least a second portion of the frequency-domain motion data associated with acceleration in a second direction that is orthogonal to the first direction.

12. The method of claim 11, wherein identifying the at least one unintentional leak based at least in part on the frequency-domain motion data includes determining location information for each of the at least one unintentional leak based at least in part on the first portion of the frequency-domain motion data and the second portion of the frequency-domain motion data.

13. The method of claim 1, wherein analyzing the motion data includes:
determining average motion data from the motion data, the average motion data indicative of an average orientation of the user interface with respect to the face of the user;
identifying a deviation in orientation of the user interface from the average orientation based at least in part on the motion data, the deviation being greater than a threshold value; and
identifying the at least one unintentional leak based at least in part on the identified deviation.

14. The method of claim 1, wherein analyzing the motion data to identify leak data includes:
extracting a plurality of motion data features from the motion data, including at least i) a user interface orientation displacement feature, and ii) a frequency-domain motion deviation feature;
identifying the at least one unintentional leak based at least in part on the user interface orientation displacement feature and the frequency-domain motion deviation feature.

15. A system comprising:
a control a control system comprising one or more processors; and
a memory having stored thereon machine readable instructions;
wherein the control system is coupled to the memory, and the method of claim 1 is implemented when the machine executable instructions in the memory are executed by at least one of the one or more processors of the control system.

16. A computer program product embodied on a non-transitory computer readable medium and comprising instructions which, when executed by a computer, cause the computer to carry out the method of claim 1.

17. A system comprising:
one or more motion sensors coupled to a user interface worn by a user during a sleep session, the user interface fluidly coupled to a respiratory therapy device for providing a flow of air from the respiratory therapy device to a respiratory system of the user;
one or more processors; and
a non-transitory computer-readable storage medium containing instructions which, when executed on the one or more processors, cause the one or more processors to perform operations including:
receiving, at a computing device, motion data associated with orientation of the user interface;
analyzing the motion data to identify leak data, the leak data indicative of at least one unintentional leak from the user interface; and
generating a notification based at least in part on the leak data, the notification indicative of the presence of the at least one unintentional leak.

18. The system of claim 17, wherein the one or more motion sensors includes one or more accelerometers, wherein receiving the motion data includes receiving the motion data from the one or more accelerometers, and wherein the one or more accelerometers includes i) an accelerometer directly coupled to the user interface; ii) an accelerometer coupled to a connector coupled to the user interface; iii) an accelerometer coupled to a conduit coupled to the user interface; or iv) any combination of i-iii.

19. The system of claim 17, wherein the operations further include:
receiving additional sensor data indicative of relative movement of the user interface with respect to the one or more accelerometers during the sleep session, wherein analyzing the motion data to identify the leak data is based at least in part on the additional sensor data.

20. The system of claim 19, wherein the additional sensor data is light sensor data of an encoded visual element associated with the user interface, wherein the light sensor data is indicative of the orientation of the user interface.

21. The system of claim 17, wherein analyzing the motion data to identify the leak data includes identifying the at least one unintentional leak when the motion data deviates from a baseline signal by at least a threshold value, wherein the baseline signal is based at least in part on (i) a portion of historical motion data assumed to be associated with low leakage or no leakage, the historical motion data associated with a prior sleep session; (ii) an extracted first portion of the motion data assumed to be associated with low leakage or no leakage; or (iii) both (i) and (ii).

22. The system of claim 17, wherein analyzing the motion data includes:
extracting frequency-domain motion data from the motion data, the frequency-domain motion data including (i) frequency-domain linear acceleration data; (ii) frequency-domain rotational acceleration data; (iii) or (i) and (ii); and
identifying the at least one unintentional leak based at least in part on the frequency-domain motion data.

23. The system of claim 22, wherein the frequency-domain motion data includes at least a first portion of the frequency-domain motion data associated with acceleration in a first direction and at least a second portion of the frequency-domain motion data associated with acceleration in a second direction that is orthogonal to the first direction.

24. The system of claim 17, wherein analyzing the motion data includes:
determining average motion data from the motion data, the average motion data indicative of an average orientation of the user interface with respect to the face of the user;
identifying a deviation in orientation of the user interface from the average orientation based at least in part on the motion data, the deviation being greater than a threshold value; and
identifying the at least one unintentional leak based at least in part on the identified deviation.

25. The system of claim 24, wherein analyzing the motion data to identify leak data includes:
extracting a plurality of motion data features from the motion data, including at least i) a user interface orientation displacement feature, and ii) a frequency-domain motion deviation feature;
identifying the at least one unintentional leak based at least in part on the user interface orientation displacement feature and the frequency-domain motion deviation feature.
